(19) United States
(12) Patent Application Publication
(10) Pub. No.: US 2026/0325854 A1
(43) Pub. Date: Oct. 22, 2026

(54) PACED COMPLEX TEMPLATE MATCH

(71) Applicant: Cardiac Pacemakers, Inc., St. Paul, MN (US)

(72) Inventor: Jessica Burr, Boston, MA (US)

(21) Appl. No.: 19/648,733

(22) Filed: Apr. 15, 2026

Related U.S. Application Data
(60) Provisional application No. 63/789,570, filed on Apr. 16, 2025.

(51) Int. Cl. A61N 1/372; A61N 1/365; A61N 1/368; A61N 1/37

(52) U.S. Cl. CPC A61N 1/37264; A61N 1/36578; A61N 1/36585; A61N 1/3682; A61N 1/37; A61N 1/37235; A61N 1/37258

(57) ABSTRACT

Systems and methods to enable remote optimization of pacing parameters to optimize resources of an ambulatory medical device are disclosed, including capturing a reference paced template for a patient based on received physiologic information with respect to a first set of pacing parameters, comparing physiologic information from an ambulatory paced beat to the reference paced template to determine a difference between the physiologic information from the ambulatory paced beat and the reference paced template, and providing a control signal to control or adjust a process or a function of the ambulatory medical device based on the difference between the physiologic information from the ambulatory paced beat and the reference paced template and a match threshold to optimize resources of the ambulatory medical device.

### CROSS REFERENCE TO RELATED APPLICATIONS

This application claims priority to U.S. Provisional Patent Application No. 63/789,570 entitled “PACED COMPLEX TEMPLATE MATCH,” filed Apr. 16, 2025, which is hereby incorporated by reference in its entirety.

### TECHNICAL FIELD

This document relates generally to medical devices and more particularly to systems and methods for confirming parameter settings in an ambulatory medical device using a template of a paced complex.

### BACKGROUND

The field of cardiac rhythm management has evolved significantly in recent years with advancements in pacing delivery locations and techniques. Modern cardiac pacing systems must accommodate an expanding array of specific cardiac locations for pacing delivery including the right ventricular septum, His bundle, left bundle branch, left bundle fascicles, right bundle branch, left ventricular septum, and coronary sinus with its associated branches. This diversification of pacing sites has created new challenges in maintaining optimal pacing parameters across these various anatomical locations.

With the increasing prevalence of remote monitoring capabilities, there exists a pressing need for automated device management systems that can maintain optimal pacing parameters without requiring frequent in-person clinical visits. Current cardiac rhythm management devices lack sophisticated algorithms for automatically optimizing and maintaining pacing parameters, particularly in the context of conduction system pacing therapy. The complexity of managing multiple lead combinations and pacing locations presents significant challenges for device follow-up and programming optimization.

Conventional approaches require frequent in-person clinical visits to adjust device parameters, which is particularly burdensome for patients with non-traditional combinations of pacing lead positions. This limitation represents a significant gap in current cardiac rhythm management technology that must be addressed to improve patient care and clinical efficiency.

### SUMMARY

Systems and methods to enable remote optimization of pacing parameters to optimize resources of an ambulatory medical device are disclosed, including capturing a reference paced template for a patient based on received physiologic information with respect to a first set of pacing parameters, comparing physiologic information from an ambulatory paced beat to the reference paced template to determine a difference between the physiologic information from the ambulatory paced beat and the reference paced template, and providing a control signal to control or adjust a process or a function of the ambulatory medical device based on the difference between the physiologic information from the ambulatory paced beat and the reference paced template and a match threshold to optimize resources of the ambulatory medical device.

An example of subject matter (e.g., a system to enable remote optimization of pacing parameters to optimize resources of an ambulatory medical device) may comprise means for receiving physiologic information from a patient, means for capturing a reference paced template for the patient based on the received physiologic information and information from a clinician during an in-clinic programming session with respect to a first set of pacing parameters, and means for detecting an ambulatory paced beat using the received physiologic information subsequent to the in-clinic programming session, comparing physiologic information from the ambulatory paced beat to the reference paced template to determine a difference between the physiologic information from the ambulatory paced beat and the reference paced template, and providing a control signal to control or adjust a process or a function of the ambulatory medical device based on the difference between the physiologic information from the ambulatory paced beat and the reference paced template and a match threshold to optimize resources of the ambulatory medical device.

In an example, which may be combined with any one or more examples described herein, the pacing parameters optionally comprise conduction system pacing parameters and the first set of pacing parameters optionally comprises a first set of conduction system pacing parameters.

In an example, which may be combined with any one or more examples described herein, the means for receiving physiologic information from the patient optionally comprise a signal receiver circuit configured to receive physiologic information from the patient, the means for capturing the reference paced template for the patient optionally comprises an assessment circuit configured to capture the reference paced template for the patient based on the received physiologic information and information from the clinician during an in-clinic programming session with respect to the first set of pacing parameters, and means for detecting the ambulatory paced beat, comparing the physiologic information from the ambulatory paced beat to the reference paced template, and providing the control signal to control or adjust the process or the function of the ambulatory medical device optionally comprises the assessment circuit configured to detect the ambulatory paced beat using the received physiologic information subsequent to the in-clinic programming session, compare physiologic information from the ambulatory paced beat to the reference paced template to determine the difference between the physiologic information from the ambulatory paced beat and the reference paced template, and provide the control signal to control or adjust the process or the function of the ambulatory medical device based on the difference between the physiologic information from the ambulatory paced beat and the reference paced template and the match threshold to optimize resources of the ambulatory medical device.

In an example, which may be combined with any one or more examples described herein, the subject matter may optionally comprise the ambulatory medical device, wherein the ambulatory medical device optionally comprises the signal receiver circuit and the assessment circuit, wherein the ambulatory medical device optionally includes an implantable medical device configured to deliver conduction system pacing to the patient, wherein to provide the control signal to control or adjust the process or the function of the ambulatory medical device optionally includes to control or adjust a conduction system pacing parameter including at least one of a voltage, atrioventricular delay, polarity, or pulse width of a conduction system pacing stimulus.

In an example, which may be combined with any one or more examples described herein, the signal receiver circuit of the implantable medical device is optionally configured to receive the physiologic information from an electrode coupled to the implantable medical device, wherein the electrode is optionally configured to sense cardiac electrical information from a heart of the patient and to deliver the conduction system pacing stimulus to a conduction pathway of the heart of the patient.

In an example, which may be combined with any one or more examples described herein, the physiologic information optionally includes at least one of nearfield or farfield cardiac electrical information or cardiac acceleration information of the heart of the patient.

In an example, which may be combined with any one or more examples described herein, the assessment circuit optionally comprises or is coupled to a therapy circuit configured to generate a pacing stimulus and a memory circuit configured to store information, including the reference paced template, wherein the reference paced template optionally comprises at least one of farfield cardiac electrical information, nearfield cardiac electrical information, cardiac acceleration information, a relationship between different cardiac electrical information, a relationship between different cardiac acceleration information, a relationship between cardiac electrical and cardiac acceleration information, or a relationship between pacing information and one or more of cardiac electrical information and cardiac acceleration information, wherein cardiac acceleration information optionally comprises a heart sound including at least one of an S1, S2, S3, or S4 heart sound.

In an example, which may be combined with any one or more examples described herein, the reference paced template optionally includes a morphology of the cardiac electrical information.

In an example, which may be combined with any one or more examples described herein, the cardiac electrical information optionally includes at least one of atrial information or ventricular information.

In an example, which may be combined with any one or more examples described herein, to determine the difference between the physiologic information from the ambulatory paced beat and the reference paced template, the assessment circuit is optionally configured to determine a correlation between the physiologic information from the ambulatory paced beat and the reference paced template or determine a difference between a feature of the physiologic information from the ambulatory paced beat and a corresponding feature of the reference paced template.

In an example, which may be combined with any one or more examples described herein, to control or adjust the process or the function of the ambulatory medical device, the assessment circuit is optionally configured to adjust a conduction system pacing parameter of the ambulatory medical device based on the difference between the physiologic information from the ambulatory paced beat and the reference paced template exceeding the match threshold and maintain the conduction system pacing parameter of the ambulatory medical device based on the difference between the physiologic information from the ambulatory paced beat and the reference paced template being within the match threshold.

In an example, which may be combined with any one or more examples described herein, to adjust the conduction system pacing parameter, the assessment circuit is optionally configured to adjust, sequentially a voltage of a conduction system pacing stimulus over a number of respective beats to determine an adjusted voltage resulting in a match between physiologic information of the respective beat and the reference paced template based on a difference between the physiologic information of the respective beat and the reference paced template being within the match threshold and for each voltage resulting in the match, an atrioventricular delay of the conduction system pacing stimulus over a number of respective beats to determine an adjusted atrioventricular delay resulting in a match between physiologic information of the respective beat and the reference paced template based on a difference between the physiologic information of the respective beat and the reference paced template within the match threshold, wherein the assessment circuit is optionally configured to program the voltage and atrioventricular delay having a smallest difference between the physiologic information of the respective beat and the reference paced template.

In an example, which may be combined with any one or more examples described herein, to adjust the conduction system pacing parameter, the assessment circuit is optionally configured to adjust, sequentially a voltage, a polarity, and a pulse width of a conduction system pacing stimulus over a number of respective beats to determine pacing parameters resulting in a match between physiologic information of the respective beat and the reference paced template based on a difference between the physiologic information of the respective beat and the reference paced template within the match threshold, wherein the assessment circuit is optionally configured, for each set of pacing parameters resulting in the match, to adjust an atrioventricular delay of the conduction system pacing stimulus over a number of respective beats to determine an adjusted atrioventricular delay resulting in a match between physiologic information of the respective beat and the reference paced template based on a difference between the physiologic information of the respective beat and the reference paced template within the match threshold, wherein the assessment circuit is optionally configured to program the pacing parameters having a closest match between the physiologic information of the respective beat and the reference paced template.

An example of subject matter (e.g., a system to enable remote optimization of pacing parameters for conduction system pacing to optimize resources of an ambulatory medical device) may comprise a signal receiver circuit configured to receive physiologic information from a patient and an assessment circuit configured to capture a reference paced template for the patient based on the received physiologic information and information from a clinician during an in-clinic programming session with respect to a first set of conduction system pacing parameters, detect an ambulatory paced beat using the received physiologic information subsequent to the in-clinic programming session, compare physiologic information from the ambulatory paced beat to the reference paced template to determine a difference between the physiologic information from the ambulatory paced beat and the reference paced template, and provide a control signal to control or adjust a process or a function of the ambulatory medical device based on the difference between the physiologic information from the ambulatory paced beat and the reference paced template and a match threshold to optimize resources of the ambulatory medical device.

An example of subject matter (e.g., a method to enable remote optimization of pacing parameters for conduction system pacing to optimize resources of an ambulatory medical device) may comprise receiving physiologic information from a patient using a signal receiver circuit, capturing, using an assessment circuit, a reference paced template for the patient based on the received physiologic information and information from a clinician during an in-clinic programming session with respect to a first set of conduction system pacing parameters, detecting, using the assessment circuit, an ambulatory paced beat using the received physiologic information subsequent to the in-clinic programming session, comparing, using the assessment circuit, physiologic information from the ambulatory paced beat to the reference paced template to determine a difference between the physiologic information from the ambulatory paced beat and the reference paced template, and providing, using the assessment circuit, a control signal to control or adjust a process or a function of the ambulatory medical device based on the difference between the physiologic information from the ambulatory paced beat and the reference paced template and a match threshold to optimize resources of the ambulatory medical device.

In an example, which may be combined with any one or more examples described herein, providing the control signal to control or adjust the process or the function of the ambulatory medical device optionally includes controlling or adjusting a conduction system pacing parameter including at least one of a voltage, atrioventricular delay, polarity, or pulse width of a conduction system pacing stimulus, wherein the signal receiver circuit and the assessment circuit are components of an implantable medical device configured to deliver conduction system pacing to the patient, wherein receiving the physiologic information from the patient optionally comprises receiving the physiologic information from an electrode coupled to the implantable medical device, wherein the electrode is optionally configured to sense cardiac electrical information from a heart of the patient and to deliver the conduction system pacing stimulus to a conduction pathway of the heart of the patient.

In an example, which may be combined with any one or more examples described herein, the reference paced template optionally comprises at least one of farfield cardiac electrical information, nearfield cardiac electrical information, cardiac acceleration information, a relationship between different cardiac electrical information, a relationship between different cardiac acceleration information, a relationship between cardiac electrical and cardiac acceleration information, or a relationship between pacing information and one or more of cardiac electrical information and cardiac acceleration information, wherein cardiac acceleration information optionally comprises a heart sound including at least one of an S1, S2, S3, or S4 heart sound.

In an example, which may be combined with any one or more examples described herein, determining the difference between the physiologic information from the ambulatory paced beat and the reference paced template optionally includes determining a correlation between the physiologic information from the ambulatory paced beat and the reference paced template or determining a difference between a feature of the physiologic information from the ambulatory paced beat and a corresponding feature of the reference paced template.

In an example, which may be combined with any one or more examples described herein, controlling or adjusting the process or the function of the ambulatory medical device optionally comprises adjusting a conduction system pacing parameter of the ambulatory medical device based on the difference between the physiologic information from the ambulatory paced beat and the reference paced template exceeding the match threshold and maintaining the conduction system pacing parameter of the ambulatory medical device based on the difference between the physiologic information from the ambulatory paced beat and the reference paced template being within the match threshold.

In an example, which may be combined with any one or more examples described herein, adjusting the conduction system pacing parameter optionally comprises adjusting, sequentially a voltage of a conduction system pacing stimulus over a number of respective beats to determine an adjusted voltage resulting in a match between physiologic information of the respective beat and the reference paced template based on a difference between the physiologic information of the respective beat and the reference paced template being within the match threshold and for each voltage resulting in the match, an atrioventricular delay of the conduction system pacing stimulus over a number of respective beats to determine an adjusted atrioventricular delay resulting in a match between physiologic information of the respective beat and the reference paced template based on a difference between the physiologic information of the respective beat and the reference paced template within the match threshold, wherein the method optionally includes programming the voltage and atrioventricular delay having a smallest difference between the physiologic information of the respective beat and the reference paced template.

In an example, which may be combined with any one or more examples described herein, adjusting the conduction system pacing parameter to optimize resources of an ambulatory medical device optionally comprises adjusting, sequentially a voltage, a polarity, and a pulse width of a conduction system pacing stimulus over a number of respective beats to determine pacing parameters resulting in a match between physiologic information of the respective beat and the reference paced template based on a difference between the physiologic information of the respective beat and the reference paced template within the match threshold, wherein the method optionally includes, for each set of pacing parameters resulting in the match, adjusting an atrioventricular delay of the conduction system pacing stimulus over a number of respective beats to determine an adjusted atrioventricular delay resulting in a match between physiologic information of the respective beat and the reference paced template based on a difference between the physiologic information of the respective beat and the reference paced template within the match threshold, wherein the method optionally includes programming the pacing parameters having a closest match between the physiologic information of the respective beat and the reference paced template.

An example of subject matter (e.g., a system to enable remote optimization of pacing parameters for conduction system pacing to optimize resources of an ambulatory medical device) may comprise a signal receiver circuit configured to receive physiologic information from a patient and an assessment circuit configured to capture a reference paced template for the patient based on the received physiologic information and information from a clinician during an in-clinic programming session with respect to a first set of conduction system pacing parameters, detect ambulatory paced beats in response to conduction system pacing using the received physiologic information subsequent to the in-clinic programming session, detect a template match loss between at least one ambulatory paced beat and the reference paced template based on a difference between physiologic information from the at least one ambulatory paced beat and the reference paced template exceeding a match threshold, and automatically provide a control signal to control or adjust a process or a function of the ambulatory medical device based on the detected template match loss, wherein to control or adjust the process or the function optionally comprises to provide an alert of the detected match loss, to transition operation of the ambulatory medical device, or to adjust at least one conduction system pacing parameter to restore template match to the reference paced template within the match threshold to optimize resources of the ambulatory medical device.

An example of subject matter (e.g., a method to enable remote optimization of pacing parameters for conduction system pacing to optimize resources of an ambulatory medical device) may comprise receiving physiologic information from a patient using a signal receiver circuit, capturing, using an assessment circuit, a reference paced template for the patient based on the received physiologic information and information from a clinician during an in-clinic programming session with respect to a first set of conduction system pacing parameters, detecting, using the assessment circuit, ambulatory paced beats in response to conduction system pacing using the received physiologic information subsequent to the in-clinic programming session, detecting, using the assessment circuit, a template match loss between at least one ambulatory paced beat and the reference paced template based on a difference between physiologic information from the at least one ambulatory paced beat and the reference paced template exceeding a match threshold, and automatically providing, using the assessment circuit, a control signal to control or adjust a process or a function of the ambulatory medical device based on the detected template match loss, wherein to control or adjust the process or the function optionally comprises to provide an alert of the detected match loss, to transition operation of the ambulatory medical device, or to adjust at least one conduction system pacing parameter to restore template match to the reference paced template within the match threshold to optimize resources of the ambulatory medical device.

In an example, a system, method, or apparatus may optionally combine any portion or combination of any portion of any one or more of the examples described herein, may optionally combine any portion or combination of any portion of any one or more of the examples described herein to comprise “means for” performing any portion of any one or more of the functions, operations, or methods of the examples described herein, or at least one “non-transitory machine-readable medium” including instructions that, when performed by a machine, cause the machine to perform any portion of any one or more of the functions or methods of the examples described herein.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example physiologic information.

FIGS. 2-3 illustrate example methods.

FIG. 4 illustrates an example of a table of parameter settings and a reduction of different combinations of parameter settings.

FIG. 5 illustrates a diagram of an example machine learning model.

FIG. 6 illustrates an example medical device system.

FIG. 7 illustrates an example patient management system.

FIGS. 8-9 illustrate example implantable medical device systems.

FIG. 10 illustrates example remote patient management or programming systems.

FIG. 11 illustrates an example machine upon which any one or more of the techniques discussed herein may perform.

### DETAILED DESCRIPTION

The heart is an electro-mechanical system performing two major pumping functions at the center of the circulatory system. The left portions of the heart, including the left atrium (LA) and left ventricle (LV), draw oxygenated blood from the lungs and pump it to the organs of the body to provide the organs with their metabolic needs for oxygen. The right portions of the heart, including the right atrium (RA) and right ventricle (RV), draw deoxygenated blood from the body organs and pump it to the lungs where the blood gets oxygenated. These pumping functions result from contractions of the myocardium (cardiac muscles).

The sinoatrial (SA) node is the natural pacemaker of a healthy functioning heart, generating electrical pulses, called action potentials, which propagate through natural electrical conduction pathways to various regions of the heart to excite the myocardial tissue of the heart. Action potentials originated from the SA node propagate through the atrioventricular (AV) node, Bachmann's bundle, the His bundle (also known as Bundle of His), the left and right bundle branches, the left bundle fascicles, and Purkinje fibers to reach the ventricular myocardium, resulting in coordinated contractions in both ventricles. Coordinated delays in the propagation of the action potentials in a normal electrical conduction system cause the various portions of the heart to contract in synchrony to result in efficient pumping functions. A blocked or otherwise abnormal electrical conduction or deteriorated myocardium can cause dyssynchronous contraction of the heart, resulting in poor hemodynamic performance, including a diminished blood supply to the heart and the body.

Artificial cardiac pacing systems have developed to rectify cardiac dyssynchrony and improve hemodynamic performance. Cardiac rhythm management (CRM) includes pacing via electrodes implanted in the apex of the RV are used in both single ventricular and biventricular (BiV) pacing. RV apex pacing directly excites the ventricular myocardium. BiV pacing typically involves RV pacing via one lead and LV pacing via another lead positioned through the coronary sinus and into a vein on the left ventricular wall can improve or restore substantially simultaneous contraction of both ventricles. Conventional cardiac pacing, including RV apex pacing and BiV pacing, often rely on propagation of activation sequences through the working myocardium, resulting in a less coordinated activation sequences than a healthy cardiac depolarization through the intrinsic normal electrical conduction system. Additionally, long-term pacing can negatively impact myocardial perfusion and structure over time.

In contrast to conventional cardiac pacing, conduction system pacing targets action potentials through the natural conduction pathways, including His bundle pacing, pacing at the left or right ventricular septum, pacing in branches of the coronary sinus or other locations adapted to capture the natural cardiac conduction system in the ventricles, which can propagate an activation signal about four times faster than a working myocardium. Conduction system pacing, including His bundle pacing, utilizes the natural conduction system, including the left and right bundle branches and Purkinje fibers, and produce efficient and coordinated cardiac response, reducing potentially long-term harmful hemodynamic effects of conventional cardiac pacing and potentially mitigating existing bundle branch block, such as disclosed in the commonly assigned Casavant et al. U.S. application Ser. No. 16/174,816, titled “Systems and Methods for His-Bundle Pacing,” herein incorporated by reference in its entirety, including its disclosure of various His bundle pacing systems, methods, and parameters.

However, conduction system pacing provides unique challenges, including specific implant locations and lead configurations, and device programming challenges, such as due to the proximity to different anatomy, exposure to different electrical signals at different times in contrast to conventional pacing. Ineffective artificial cardiac pacing targeting the natural specialized cardiac conduction system may result in a complex or less efficient or effective myocardial contraction than conventional pacing. Further, many existing pulse generators designed for conventional pacing lack conduction system pacing algorithms capable of automatically adjusting and optimizing pacing parameters.

The present inventor has recognized, among other things, improved systems and methods for monitoring and maintaining optimal cardiac pacing parameters through template matching and automated parameter adjustment. The systems and methods presented herein address the technical challenges associated with maintaining optimal pacing parameters over time by implementing a comprehensive reference paced template matching process to automatically and remotely monitor and adjust pacing parameters to maintain consistent cardiac activation patterns in an ambulatory setting. In certain examples, comparing a reference paced template to subsequent paced beats can provide an additional level of safety and control over remote and in-clinic patient monitoring and programming, reprogramming, or adjusting pacing parameters, enabling automatic maintenance and adjustment of optimal pacing without requiring frequent in-person clinical visits.

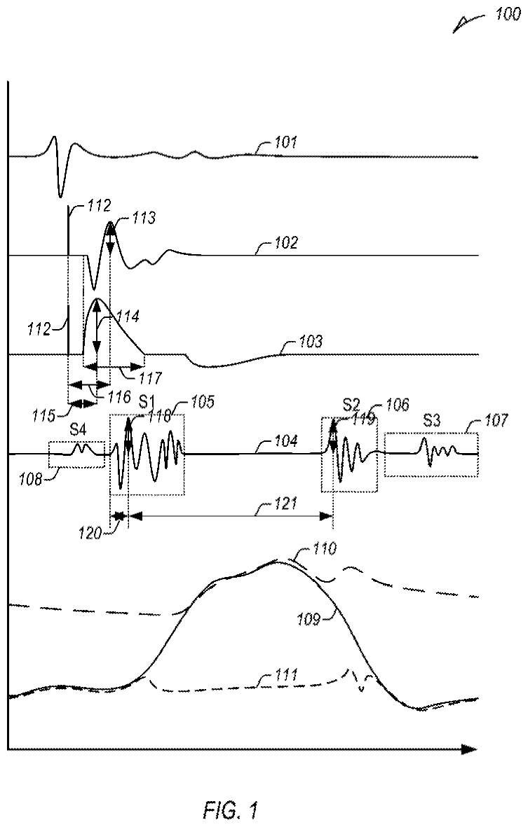

An ambulatory medical device can capture a template of a paced complex during an in-clinic programming session using a combination of implanted lead signals, interelectrode timing measurements, and device sensor data. For example, during device programming in an in-clinic setting, a clinician can perform a series of programming and testing protocols to determine optimal settings for the patient, given lead placement, pacing thresholds, hardware, etc. The ambulatory medical device can receive an indication, such as from the clinician determining settings or from or through a corresponding device (e.g., a medical device programmer, etc.) or process, such as a programmed schedule, etc., and in response, capture the template of the paced complex corresponding to the settings programmed by the clinician at the time of programming. In other examples, pacing parameters programmed in a session initiated via magnetic coupling between a medical device programmer and the ambulatory medical device can trigger capture and storage of both resulting parameter settings from the session as established during the programming session initiated via the magnetic coupling and the reference paced template in response to said parameter settings. The reference paced template can include one or more of electrograms or physiologic information of the patient corresponding to a paced beat, such as the information illustrated in FIG. 1 or combinations thereof, in certain examples, stored by the ambulatory medical device, such as in a memory circuit, etc. Subsequent paced beats can be compared to the reference paced template during ambulatory monitoring to determine if subsequent pacing at existing pacing parameters maintains sufficient capture as compared to that determined by the clinician to be safe and effective. When deviations from the stored template are detected, the system can automatically adjust pacing parameters including one or more of voltage, pulse width, polarity, or atrioventricular delay (AVD) to maintain or remotely restore pacing to in-clinic quality and efficacy. Such adjustments improve device efficiency, ensuring optimal use of the ambulatory medical device and increasing efficient use of its usable life.

Although described herein as largely atrioventricular delay, in other examples, one or more other cross-chamber delays can be adjusted depending on the type of pacing (e.g., conduction system pacing, or the specific type of conduction system pacing, etc.), whether occurring in the right ventricle, the left ventricle, the coronary sinus, etc. In such examples, the cross-chamber delay can include, in certain examples in addition to or instead of an atrioventricular delay, one or more of a ventricular-ventricular delay, atrial-HIS (or other conduction system pacing) delay, etc.

Multiple signal characteristics can be captured to form a reference paced template, including farfield and nearfield electrogram (EGM) signals or signal morphology aspects (e.g., width, peaks, frequency response, rate of change, etc.) from different vectors or locations, intervals between pacing impulses and electrogram peaks, relationships between nearfield and farfield electrograms from the same and different implanted leads, and relationships between pacing impulses and cardiac acceleration information, such as heart sounds (e.g., S1, S2, S3, S4, etc.) detected by an accelerometer, etc. This multifaceted approach provides a robust template that accounts for the complex electrical and mechanical aspects of cardiac activation.

The reference paced template can provide significant improvements to remote ambulatory patient therapy and monitoring, for example, by providing a clear and clinically significant test to determine when intervention is required, such as by detecting a deviation between detected paced beats and the reference paced template, but also providing a goal for remote pacing parameter adjustments that result in a pacing response that matches a previous patient response determined by a clinician to be both clinically safe and effective for that patient, optimizing efficient use of the ambulatory medical device for the patient, or providing an indication that clinical intervention is required to maintain optimal efficacy. A methodical approach to parameter adjustment can be employed, systematically modifying, voltage, polarity, pulse width, and cross-chamber delays (e.g., AVD, VVD, etc.) according to programmable, and in certain examples, sequential criteria to restore reference paced template matching while maintaining appropriate safety margins (e.g., ensuring that a voltage above the match voltage can be provided to maintain pacing capture), providing a significant advancement in cardiac rhythm management technology by enabling automated, remote optimization of pacing parameters, particularly for conduction system pacing applications, thereby reducing the need for in-person clinical visits, ensuring optimized device usage while maintaining safe and effective therapeutic outcomes.

Previous systems and methods performed remote capture verification, such as by detection of an evoked response or a percentage of paced beats or changes in parameters, such as pacing thresholds generally, etc., such as described in the commonly assigned Burr et al. U.S. application Ser. No. 18/652,922, titled “Conduction System Pacing Optimal Output Setting Indicator,” herein incorporated by reference in its entirety, including its disclosure of capture confirming criteria and automatic pacing threshold test, etc. However, the additional verification of determining capture not as function of general measures or changes, but as a comparison to specific previously identified and captured information (e.g., electrograms, morphology, etc.) in an in-clinic setting with respect to the specific patient provides an additional level of safety and remote evaluation of changes in patient status that are particular and individualized for that specific patient. Additionally, when making remote adjustments or programming changes, having the reference paced template (e.g., electrogram, morphology, heart sounds, physiologic information associated therewith, etc.) to re-attain, capture, or match provides an additional level of safety and efficacy not otherwise attainable remotely with generic or more relative measures, as particular electrograms deemed safe and sufficient by a clinician are available for verification of sufficient performance and quality. Here, remote parameter adjustments can be made safely and effectively, where an in-clinic follow-up may previously have been required.

FIG. 1 illustrates an example relationship 100 between measured patient physiologic information over at least a portion of one or more cardiac cycles, including an atrial electrogram 101 (including a P wave) and a ventricular electrogram 102 (including a QRS complex and the R wave) and an evoked response 103 in response to a pacing stimulus 112, such as from different nearfield or farfield vectors, electrodes, etc. FIG. 1 additionally illustrates cardiac acceleration 104 (e.g., a heart sound signal including a first heart sound (S1) 105, a second heart sound (S2) 106, a third heart sound (S3) 107, and a fourth heart sound (S4) 108), left ventricular pressure 109, aortic pressure 110, and left atrial pressure 111. electrocardiogram signal) over at least a portion of one or more cardiac cycles.

The horizontal axis in FIG. 1 is time, shown without scale, as the time of the cardiac cycle depends upon the heart rate of the patient, which greatly varies (e.g., typically between 60 and 100 beats per minute (bpm), but more or less in certain examples). Systolic time intervals, such as pre-ejection period (PEP) or left ventricular ejection time (LVET) can be indicative of clinically relevant information, including contractility, arrhythmia, Q-T prolongation (with electrogram information), etc. The pre-ejection period can be measured from a Q wave of an electrogram to the time of the aortic valve opening. The left ventricular ejection time can include a time between the aortic valve opening and the aortic valve closing. In other examples, one or more systolic time intervals can be detected and used to detect physiologic information of a subject (e.g., PEP/LVET, one or more mechanical, electrical, or mechanical-electrical time intervals, etc.).

In an example, the reference paced template information can include combinations or permutations of one or more of: the ventricular electrogram 102, an amplitude of an R wave peak 113, a first time period 116 between the pacing stimulus 112 and the R wave peak 113, the evoked response 103, an amplitude of an evoked response peak 114, an evoked response width 117, a second time period 115 between the pacing stimulus 112 and the evoked response peak 114, an amplitude of an S1 peak 118, an amplitude of an S2 peak 119, a third time period 120 between the R wave peak 113 and the S1 peak 118, or a fourth time period 121 between the S1 peak 118 and the S2 peak 119, morphologies of different features, time periods between different measurements or features, ratios of different measurements, features, or time periods, etc. In other examples, frequency response,

In certain examples, the combination of cardiac acceleration and cardiac electric information or cardiac electrical information from both nearfield and farfield capture can be used to supplement local or nearfield cardiac electrical information to address situations such as fusion, where multiple chambers are captured simultaneously, pseudofusion, where a local response may appear to have evoked capture of a portion of the myocardium, however, not effecting capture of a full chamber, or one or more other conditions, etc.

### Method Examples

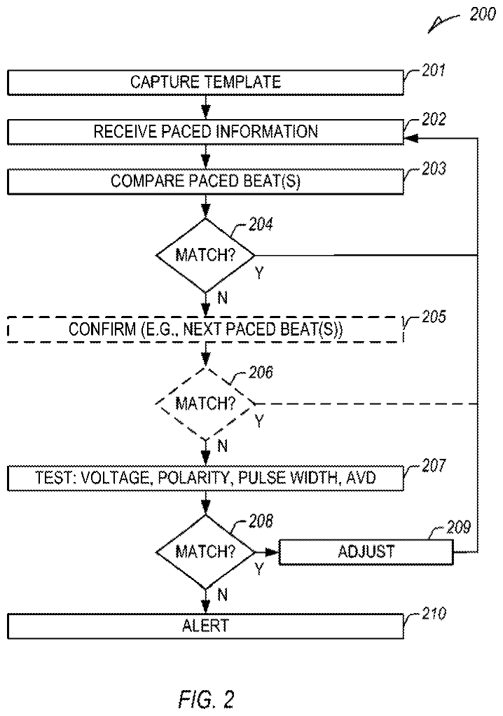

FIG. 2 illustrates an example method 200 to enable remote template-based monitoring and optimization of conduction system or other pacing parameters for a patient using an ambulatory medical device, such as an implantable medical device configured to provide conduction system pacing therapy, etc. Although described herein with respect to conduction system pacing, in other examples, the systems and methods described herein can be used with other pacing therapies to enable or assist remote monitoring and optimization of pacing parameters.

At step 201, a reference paced template for a patient can be captured, such as using an assessment circuit of an ambulatory medical device or one or more other circuits or components. The reference paced template for the patient can include, among other things, one or more nearfield or farfield cardiac electrical signals, including atrial, ventricular, evoked response, myocardial, or one or more other electrograms or cardiac electrical signals or components or aspects thereof, such as amplitudes, timings, widths, etc., in response to a pacing stimulus delivered by a therapy circuit, a pulse generator, the assessment circuit, or one or more other circuits or components, etc.

In an example, the reference paced template can include other physiologic information, such as cardiac mechanical information, including cardiac acceleration information, such as heart sound information, or other cardiac mechanical information such as pressure signals, etc. In other examples, the reference paced template can include other information, such as morphologies of different features, time periods between different measurements or features, ratios of different measurements, features, or time periods, such as otherwise described herein, etc.

In an example, the ambulatory medical device can store the reference paced template or information about the paced template, including parameter settings for a pacing stimulus set by the clinician in the programming session to establish the reference paced template, etc., such as using the assessment circuit, a memory circuit, or one or more other circuits or components, etc. In certain examples, the reference paced template can be received or captured in combination with one or more external devices, such as a medical device programmer, a remote patient management system, etc.

Once the reference paced template is established, the ambulatory paced beats can be compared to the reference paced template at different intervals, for example, ranging from a set number of paced beats, to hourly, to weekly, etc. The reference paced template matching process can a combination of the characteristics to determine whether current pacing is producing the desired or expected cardiac activation pattern.

At step 202, physiologic information of a patient can be received, such as using a signal receiver circuit or one or more other components. The physiologic information can include physiologic information of the patient, such as one or more types of physiologic information or parameters sensed using one or sensors of the ambulatory medical device associated with the patient, etc. In other examples, other information can be received, such as one or more parameters settings of the ambulatory medical device (e.g., existing pacing parameters, settings indicative of a state or mode of the ambulatory medical device, etc.).

At step 203, a first one or more ambulatory paced beats can be detected or determined in response to one or more pacing stimuli, such as to compare the first one or more ambulatory paced beats to the reference paced template, using the assessment circuit or one or more other circuits or components, etc. For example, when comparing an amplitude or time of one or more features, a time period between features, a ratio of amplitude or time periods, etc., such characteristics of the ambulatory paced beat can be compared to corresponding features of the reference paced template. In other examples, such as when comparing morphology between different signals, a correlation or difference between signals can be determined, such as by aligning different signals and determining the difference between the different signals at different points, etc.

At step 204, a match can be determined between the reference paced template and the first one or more ambulatory paced beats, or features thereof, such as using one or match thresholds, etc. In certain examples, a match can be determined if one or more determined differences are within one or more match thresholds. In an example, the one or more match thresholds can include patient-specific or clinical values, received by a clinician, or determined as a predetermined amounts or a threshold percentage of the reference paced template, etc. In an example, clinicians can customize template matching criteria, physiologic information associated with the reference paced template, or the one or more match thresholds used to determine the match, such as to account for diverse requirements of patients with various lead combinations and pacing locations, etc.

In certain examples, one or both of steps 203 and 204 can be performed at selected intervals, such as hourly, daily, etc. If a match is determined at step 204, such as by one or more differences being within one or more match thresholds, the method 200 can return to step 202 for the next comparison at a subsequent interval. However, if deviation from the reference paced template is detected at step 204 that exceeds the one or more match thresholds, a multi-step threshold assessment can optionally be implemented at step 205 to confirm the loss of match at a second one or more paced beats, such as the subsequent or next paced beat or set of paced beats immediately following those detected, determined, or compared at steps 203 and 204, etc.

In an example, the multi-step threshold assessment can include different assessments or determinations, e.g., having a different sensitivity or specificity or consuming or requiring different resources. For example, a first set of features can be detected and compared at step 203, such as one or more of the amplitudes or time periods illustrated in FIG. 1, etc., requiring a first amount of system resources. If no match is determined at step 204, a confirmation can be performed at step 205, in certain examples the same as at steps 203 and 204, or in other examples, requiring additional system resources than required by steps 203 and 204. For example, confirmation at step 205 can require a morphology comparison of signals, a comparison of additional features than required at step 203, additional paced beats, etc. Optional confirmation can prevent unnecessary parameter changes and alerts, such as due to isolated anomalous beats, etc.

At step 206, a match can be determined between the reference paced template and the second one or more ambulatory paced beats, or features thereof, such as using the one or match thresholds, etc. If a match is determined at step 206, the method 200 can return to step 202 for the next comparison at the subsequent interval. However, if deviation from the reference paced template is detected at step 206 that exceeds the one or more match thresholds, one or more parameter adjustments can be tested at step 207.

At 207, a test of pacing parameter adjustments can be performed, such as by the assessment circuit or one or more other circuits or components, to regain a match between the reference paced template and detected ambulatory paced beats. In an example, a control signal can be provided to control or adjust (e.g., automatically) a process or a function of the ambulatory medical device, including, for example, a sequence of conduction system pacing parameter adjustments to test one or more parameter changes to regain a match between the reference paced template and one or more subsequently detected ambulatory paced beats. In an example, one or more of voltage, polarity, pulse width, or atrioventricular delay (e.g., or other cross-chamber delay, etc.) can be adjusted.

In an example, a specific sequence can be performed, in order, until a match is determined, including: (1) voltage; (2) polarity; (3) and pulse width to regain the match between the reference paced template and ambulatory paced beats, with adjustment of atrioventricular delay after any successful determination of match to further optimize conduction system pacing parameters. In certain examples, the sequence can include atrioventricular delay as part of the sequence. However, in many examples, the programmed atrioventricular is shortened during voltage, polarity, and pulse-width testing to ensure a paced and not intrinsic beat results. In other examples, a custom sequence can be implemented by a clinician for their patients, or for a specific patient by their clinician or one or more other caregivers associated with the patient, etc., such as to take into account patient need, etc. This flexibility accommodates diverse requirements of patients with various lead combinations and pacing locations. In yet other examples, one or more other sequences can be performed in one or more different orders, such as: voltage, pulse width, atrioventricular delay; pulse width, polarity, voltage, atrioventricular delay; or one or more other combinations, permutations, or orders.

If a match is determined at step 208, an adjustment can be made at step 209, such as by the assessment circuit or one or more other circuits or components, to program the matching parameter settings, after which the method 200 can return to step 202 and continue to monitor the patient. However, if a match is not determined at step 208, parameter settings can be returned to the original settings, and a control signal can be provided at step 210, such as by the assessment circuit or one or more other circuits or components, to provide one or more of an alert to a process or a clinician or caregiver for additional review or a control signal to automatically adjust a follow-up schedule for the patient with a clinician in an in-clinic setting or to change one or more modes in the ambulatory medical device (e.g., to provide emergency backup pacing, to transition from a low-power mode to a high-power mode, to provide additional sensing, storage, communication, or monitoring, etc.), etc.

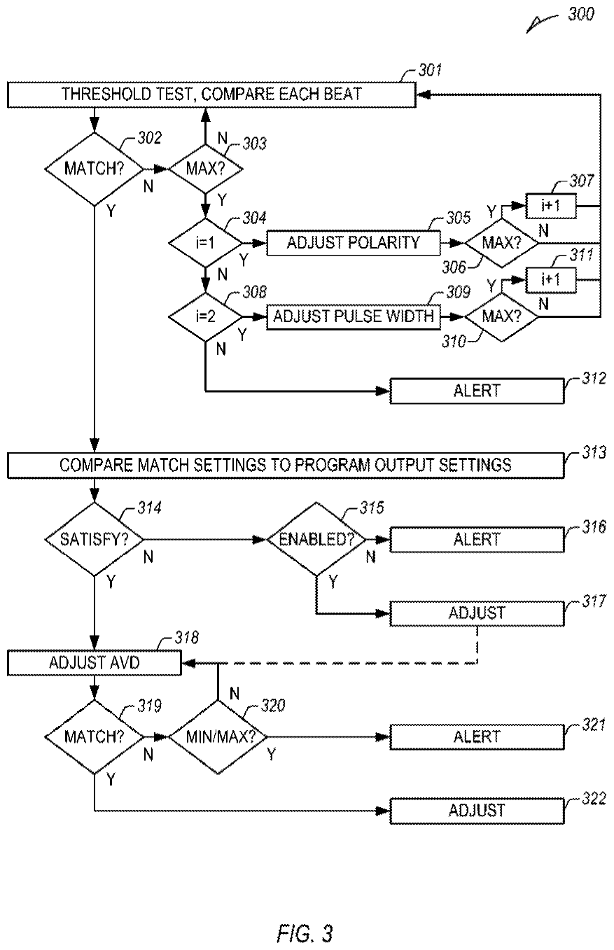

FIG. 3 illustrates an example method 300 including, in certain examples, a more specific description or implementation of the test of pacing parameter adjustment performed at step 207 in FIG. 2.

At step 301, a threshold test can be performed, such as by the assessment circuit or one or more other circuits or components, to determine voltages of pacing stimulus (e.g., conduction system pacing stimulus), delivered by a therapy circuit, a pulse generator, an assessment circuit, or one or more other circuits or components, etc., that result in capture of a paced beat, such as opposed to intrinsic activation. In an example, the threshold test can include a step-up or step-down threshold test, where different voltages within programmable ranges (e.g., 0.1-7.5 volts, etc.) are tested at different cardiac cycles.

In an example, because match was lost to arrive at the threshold test at step 301, it can be beneficial to remotely perform a step-down test, such as to ensure a fewer number of missed paced beats in contrast to a step-up test. However, in certain examples, such as if the previously programmed voltage resulting in loss of match was a lower voltage, or below a threshold (e.g., below the middle setting or voltage, in the lower-third of settings or voltages, etc.), a step-up test can be performed, in certain examples, starting at the previous setting, a setting below the previous setting, etc.

At step 302, a match can be determined between the reference paced template and captured paced beats from step 301, or features thereof, such as by the assessment circuit or one or more other circuits or components, based on the one or match thresholds, etc. If a match is determined at step 302, the method 300 can proceed to step 313. If a match is not determined at step 302, the starting voltage for the threshold test (or the ending voltage) can be evaluated at step 303.

At step 303, if the threshold test was not performed up to a maximum starting or ending voltage, such as previously taking into account a programmable safety margin, etc., the starting or ending voltage can be adjusted to a maximum (e.g., beyond the safety margin from the maximum output voltage, etc.) and the method 300 can return to step 301 to re-perform the threshold test with the higher starting or ending voltage. In an example, determination of a match at step 302 can include evaluation of whether any matches would allow for a programmable safety margin and still be within a maximum output voltage, such that, in certain examples, a matching voltage from the threshold test at step 301 that does not allow for a programmable safety margin is not a match at step 302.

In an example, if a step-up test was performed previously at step 301, it can be advantageous to perform a step-down test after adjusting the voltage to the maximum at step 303, in certain examples only stepping down to the previous highest setting before proceeding to step 304.

At step 303, if the threshold test was performed with the maximum starting or ending voltage (e.g., device maximum, a programmed maximum, a user input, etc.), or if a match is determined at step 302 using a voltage above the programmed safety margin, the method 300 can proceed to step 304 for subsequent additional adjustment to determine if a pacing stimulus satisfying output requirements can be determined (e.g., achieving a match at different parameter settings with a match voltage that allows a safety margin to be added). At step 304, if an integer i, which starts at a value of 1 at method 300, has a value of 1, the method 300 can proceed to step 305.

At step 305, a polarity of the pacing stimulus can be adjusted, such as by the assessment circuit or one or more other circuits or components. In certain examples, only two polarities are possible (e.g., from ring-to-tip to tip-to-ring, etc.), such that changing from the first polarity to the second polarity is the maximum number of polarity adjustments. In other examples, other polarities are possible (e.g., unipolar, bipolar, tip-to-ring, tip-to-coil, etc.), such as additional electrodes such that multi-cite pacing can be enabled with additional polarities or combinations of anodes and cathodes for delivering pacing stimulus at the target pacing site, such that an adjustment from a first polarity to a second polarity is not the maximum number of polarity adjustments.

At step 306, if the polarity adjustment at step 305 represents the last available polarity adjustment, for example, the maximum number of polarity adjustments (e.g., a device maximum, a programmed maximum, a maximum available number, a user input, etc.), the integer i can be incremented at step 307, such as by the assessment circuit or one or more other circuits or components, and the method 300 can proceed to step 301 to perform the threshold test again with the adjusted polarity. In this example, following the additional threshold test at step 301, if the method returns to step 304, the value of the integer i will be 2, and the method 300 can proceed to step 308. In contrast, at step 306, if the polarity adjustment at step 305 is not the last available polarity adjustment (e.g., the maximum number of polarity adjustments, etc.), the integer i is not incremented, such that method 300 can proceed to step 301 to perform the threshold test again with the adjusted polarity and, if the method returns to step 304, the value of the integer i will still be 1 and the polarity of the pacing stimulus will be adjusted again at step 305 until all available polarity adjustments have been exhausted in combination with additional threshold tests at step 301.

At step 308, if the integer i has a value of 2, the method 300 can proceed to step 309. In certain examples, before proceeding to step 309, the polarity of the pacing stimulus can be returned to the original polarity before proceeding to step 309.

At step 309, a pulse width of the pacing stimulus can be adjusted, such as by the assessment circuit or one or more other circuits or components. In certain examples, a range of pulse widths are available for the pacing stimulus (e.g., 0.1-2.0 milliseconds, etc.) and a number of different values can be tried, for example, starting from the smallest, the largest, or using one or more search algorithms or techniques (e.g., jump search, etc.) based on the previous setting or previously adjusted or tested settings, etc. In an example, the pulse width can start from the smallest available pulse width, or in other examples, from a value or setting above the previous setting, such that the adjusted pulse width larger than the previous pulse width.

At step 310, if the pulse width adjustment at step 309 represents the last available pulse width setting (e.g., a device maximum, a programmed maximum, a maximum available number, a user input, etc.), for example, the maximum setting in a linear or jump search from the shortest to longest settings, etc., the integer i can be incremented at step 311, such as by the assessment circuit or one or more other circuits or components, and the method 300 can proceed to step 301 to perform the threshold test again with the adjusted pulse width. In this example, if the method returns to step 308, following the additional threshold test at step 301, the value of the integer i will be 3, and the method 300 can proceed to step 312. In contrast, at step 310, if the pulse width adjustment at step 309 is not the last available pulse width adjustment (e.g., the maximum setting, etc.), the integer i is not incremented, such that method 300 can proceed to step 301 to perform the additional threshold test with the adjusted pulse width and, if the method returns to step 308, the value of the integer i will still be 2 and the pulse width of the pacing stimulus will be adjusted again at step 309 until all available pulse width adjustments have been exhausted in combination with additional threshold tests at step 301.

At step 312, a control signal to control or adjust a process or a function of the ambulatory medical device can be provided, such as by the assessment circuit or one or more other circuits or components. In an example, the control signal can include an alert to a clinician, including that match from the reference paced template has been lost and cannot be retained, or cannot be attained with a programmed safety margin. In other examples, the control signal can include a signal to automatically adjust a follow-up schedule for the patient, such as by a remote patient management system, etc., including scheduling a 12-lead electrocardiogram session with a clinician able to adjust or reprogram the ambulatory medical device for conduction system or other pacing. In other examples, one or more other functions or processes can be adjusted, such as a transition between different modes, such as an ambulatory mode and an alert mode, etc.

At step 313, match settings can be compared to program output settings. In certain examples, multiple voltages at different polarity or pulse width settings can match at step 302, such that there can be one or more matches. In cases of multiple matches, different sets of matching parameters can be compared to the original or previous programmed output settings, including specific pacing stimulus voltages for the respective chamber, etc. In certain examples, the one or more matches can be ordered by the voltages of each match, the amount of difference determined by the match at step 302, energy requirements of the matching polarity or pulse width, or in certain examples, combinations thereof.

At step 314, the match voltage can be compared to one or more requirements, such as by the assessment circuit or one or more other circuits or components. The one or more requirements can include, for example, if the original or previous programmed output voltage is greater than or equal to the match voltage plus a programmed safety margin, if the match voltage plus a programmed safety margin is less than or equal to a maximum output voltage, etc. If one or more requirements are not satisfied at step 314, such as if the original or previous programmed output voltage is less than one or more match voltages and a programmed safety margin, the method 300 can proceed to step 315. If the one or more requirements are satisfied at step 314, such as if the original or previous programmed output voltage is sufficient, or greater than or equal to one or more match voltages and a programmed safety margin, the method 300 can proceed to step 318.

At step 315, ambulatory change settings of the ambulatory medical device can be evaluated, such as by the assessment circuit or one or more other circuits or components. If an ambulatory change setting is off at step 315, such that ambulatory changes are not allowed, or ambulatory changes beyond the original parameter settings (e.g., set by the clinician, etc.) are not allowed, a control signal to control or adjust a process or a function of the ambulatory medical device can be provided at step 316, such as described above with respect to step 312, etc. In contrast, if an ambulatory change setting is on at step 315, such that ambulatory changes can be made for the patient outside of an in-clinic setting, or ambulatory changes beyond the original pacing and the match voltage satisfies a programmed safety margin, the parameter settings can be adjusted at step 317, such as by the assessment circuit or one or more other circuits or components, to the tested parameter settings providing the match. If multiple voltages provide a match and the programmable safety margin, the lowest matching voltage plus the programmable safety margin (e.g., a match voltage of 1.5 V plus a safety margin of 0.3 V, resulting in a pacing voltage of 1.8 V, etc.) can be selected, and the method 300 can optionally proceed to step 318.

At step 318, an atrioventricular delay of the pacing stimulus can be adjusted, such as by the assessment circuit or one or more other circuits or components. In certain examples, a range of atrioventricular delays (e.g., 30 to 400 milliseconds, etc.) are available for the pacing stimulus and a number of different values can be tried, for example, starting from the smallest, the largest, or using one or more search algorithms or techniques (e.g., jump search, etc.) based on the previous setting or previously adjusted or tested settings, etc. In an example, the atrioventricular delay can start from the smallest available atrioventricular delay, or in other examples, from a value or setting above or below the previous setting, etc.

At step 319, an ambulatory paced beat can be detected or determined in response to a pacing stimulus delivered with the adjusted atrioventricular delay from step 318, and a match between the reference paced template and a detected or determined ambulatory paced beat can be determined, such as by the assessment circuit or one or more other circuits or components, based on the one or match thresholds, etc. In an example, multiple atrioventricular delays can be tested to determine the best match to the reference paced template, or to select the atrioventricular delay having a match with the lowest setting.

If a match is not determined at step 319, the tested atrioventricular delays can be evaluated at step 320 to determine if all allowable atrioventricular delays, from a min to a max, have been evaluated, or if min and max matching atrioventricular delays have been determined. In certain examples, a range of atrioventricular delays can be allowable, such as including the value of the original parameter settings or a threshold amount above or below the original parameter settings, etc. If the full range of allowable atrioventricular delays has not been tested, or if min and max matching atrioventricular delays have not been determined, the method 300 can return to step 318. If a match is determined at step 319, and the parameter settings can be adjusted at step 322, such as by the assessment circuit or one or more other circuits or components.

Although illustrated as a series of steps above, in certain examples, one or more steps are optional, and in other examples, different combinations or permutations of these or other steps or examples can be combined to form other methods or processes, which is also applicable to other examples discussed herein. In addition, the methods described herein can be implemented in whole or in part using one or more processors and one or more memory devices storing instructions or using one or more circuits or components as described herein.

Additionally, although described herein with respect to conduction system pacing, other pacing types can similarly benefit from such reference paced template match, and various systems and methods described herein can be employed in other types of pacing to ensure match and optimization of pacing parameters.

### Ambulatory Medical Devices

Ambulatory medical devices (AMDs), including implantable, subcutaneous, wearable, insertable, or one or more other medical devices, etc., can monitor, detect, or treat various conditions, including heart failure (HF), atrial fibrillation (AF), etc. Ambulatory medical devices can include sensors to sense physiologic information from a patient and one or more circuits to detect one or more physiologic events using the sensed physiologic information or transmit sensed physiologic information or detected physiologic events to one or more remote devices. Additionally, ambulatory medical devices can be configured to provide electrical stimulation or one or more other therapies or treatments to the patient, such as to improve cardiac function, etc.

Ambulatory patient monitoring can provide early detection of worsening patient condition, including worsening heart failure, atrial fibrillation, etc. Accurate identification of patients or groups of patients at an elevated risk of future adverse events may control mode or feature selection or resource management of one or more medical devices, control notifications or messages in connected systems to various users associated with a specific patient or group of patients, organize or schedule physician or patient contact or treatment, or prevent or reduce patient hospitalization.

Correctly identifying and safely managing patient risk of worsening condition may avoid unnecessary medical interventions, extend the usable life of medical devices, and reduce healthcare costs. In addition, in situations where different operating modes, features, or therapies are available, correctly monitoring, detecting, and identifying patient status, including improving or worsening patient condition, and modifying one or more medical device functions based thereon, can improve medical device efficiency, such as by reducing unnecessary resource consumption, thereby extending the usable life of the ambulatory medical device.

Ambulatory medical devices are devices configured to be implanted in or otherwise positioned on or about patients to monitor physiologic information, such as cardiac electrical, heart sound, respiration, impedance, pressure, physical activity, or other physiologic information or one or more other physiologic parameters of the patient, or to provide electrical stimulation or one or more other therapies or treatments to optimize or control one or more body functions of the patient, such as contractions of a heart, etc. Ambulatory medical devices can include implantable or external (e.g., wearable) cardiac rhythm management devices configured to monitor or provide stimulation to the patient.

Cardiac rhythm management devices are generally configured to receive cardiac electrical information from, and in certain examples, provide electrical stimulation to, one or more electrodes located within, on, or proximate to the heart, such as coupled to one or more leads and located in one or more chambers of the heart, within the vasculature of the heart near one or more chambers, or otherwise attached to or in contact with or proximate to the heart. Cardiac rhythm management devices can include, among others, pacemakers, implantable cardioverter defibrillators (ICDs), subcutaneous implantable cardioverter defibrillators (S-ICDs), cardiac resynchronization therapy defibrillators (CRT-Ds), insertable cardiac monitors (ICMs), leadless cardiac pacemakers (LCPs), or wearable or remote monitoring systems.

Cardiac resynchronization therapy (CRT) refers generally to stimulation therapy generated and provided to one or more chambers of the heart (e.g., frequently two or more of the right ventricle (RV), the left ventricle (LV) (e.g., commonly through the cardiac vasculature), or the right atrium (RA), etc.) to improve cardiac function, such as to improve coordination of contractions between different chambers of the heart (e.g., the right ventricle and the left ventricle, the right atrium and the right ventricle, etc.) or to otherwise improve cardiac output or efficiency. Cardiac resynchronization therapy can include biventricular pacing (e.g., both right and left ventricular pacing), single-chamber pacing (e.g., right ventricle pacing, left ventricle pacing, etc.), sensing or pacing in one or more other chambers or combinations of chambers (e.g., right atria, etc.), as well as multi-site pacing (MSP) (e.g., applying one or more stimulation signals to multiple (e.g., two or more) electrodes in or proximate to a chamber (e.g., commonly the left ventricle, but also in certain examples the right ventricle, the right atrium, or combinations thereof) for a single cardiac cycle), and in certain examples, conduction system pacing, such as HIS-bundle pacing, septal pacing (e.g., from the right ventricle, the left ventricle, etc.), etc. The timing of stimulation signals in the cardiac cycle or with respect to one or more cardiac events often varies depending on a number of factors, including placement of the lead or electrodes, propagation of the stimulation signals through the tissue, and stimulation parameters, such as stimulation amplitude, type, timing, etc.

Ambulatory medical devices can provide different monitoring, storage, communication, or therapy using different modes, however, with different power and resource requirements and varying effectiveness for different patients. For example, a variety of therapy modalities are available to patients, but not all patients receive the optimal medical device, therapy mode, or therapy parameter settings at first programming. Recent literature suggests that nearly 40% of current pacing therapy is suboptimal, where suboptimal is defined as cardiac resynchronization therapy resulting in cardiac capture of one or more chambers (typically the LV for CRT patients generally) in less than 98% of cardiac beats. One common reason for suboptimal cardiac resynchronization therapy is inappropriate programming of parameter settings of medical devices (e.g., implantable medical devices) configured to provide cardiac resynchronization therapy (e.g., CRT devices). Although discussed extensively herein with respect to cardiac capture, other desired clinical outcomes are contemplated, such as selection of pacing or sensing vectors, pacing mode, resource usage associated with communication or transmission of physiologic data from the ambulatory medical device or storage of physiologic data, etc.

### Programming

Ambulatory medical devices, including implantable cardiac rhythm management, cardiac resynchronization therapy, or monitoring devices, etc., include wireless telemetry circuits and components to communicate via one or both of radio frequency (RF) telemetry or inductive telemetry with one or more other ambulatory medical or external devices, such as a remote patient management device, to enable remote programming of the ambulatory medical device or data transfer in one or more telemetry sessions. RF telemetry (e.g., short-range RF telemetry), such as Medical Implant Communication Service (MICS) (402-405 MHz frequency with a range of 2 m); Bluetooth® or Bluetooth® Low Energy (BLE) (2.4-2.483 GHz frequency with a range up to 10 m), etc., utilizes radio waves to communicate over distances up to several meters or more. In contrast, inductive telemetry utilizes electromagnetic induction to communicate over short distances, typically less than 15 cm, minimizing interferences but requiring proximity between coupled telemetry antennas.

There are generally different classes of medical device programmers and programming instructions. Certain changes that may materially impact the patient (e.g., turning on or off therapy modes, etc.) can only be performed through or separately in coordination with inductive telemetry, which requires proximity by a clinician or caregiver operating a medical device programmer having a telemetry wand in close proximity to the patient. Other changes (e.g., adjustments to parameter settings, such as values of timing intervals, amplitudes, thresholds, vectors, or other instructions, parameters, or ranges defined or previously authorized by a clinician, etc.) can be managed remotely, such as through a remote patient management system coupled to a cloud-based server through a network connection and to the ambulatory medical device through an RF telemetry (e.g., MICS, BLE, etc.).

In certain examples, programming instructions for an ambulatory medical device can be generated or received, such as programming instructions to transition from an initial (at the time of receiving the programming instructions) programming state or set of parameter settings of an ambulatory medical device to a desired programming state or set of parameter settings. For example, parameter settings in a first time period can be representative of a first cardiac rhythm management therapy, implemented at a time of implant (e.g., including a post-implant time period after a received indication of a time of implant of an ambulatory medical device, etc.) or later, such as after a recovery period or after a monitoring period after one or more previous reprogramming instructions. Parameter settings can subsequently be received in a second time period or one or more other time periods subsequent to the first time period, each representative of a specific therapy having defining characteristics, such as a mode or one or more parameter settings. and can be received from an ambulatory medical device or a remote or external device configured to program the ambulatory medical device.

In an example, if, after implementation of the programming instructions, information about the patient or the ambulatory medical device indicates a worsening patient or ambulatory medical device status, such as represented by a change in condition, status, indication, amount of change from a predicted or expected value, etc., greater than a threshold amount, the ambulatory medical device can revert or be programmed to revert to the initial programming state or set of parameter settings, such as for safety of the patient or a reduction in harm or negative impact to the ambulatory medical device, etc. However, such reversions are often determined after a recovery period (e.g., 2 weeks, one month, three months, etc.) and often require subsequent programming instructions away from the initial programming state or set of parameter settings, slowing implementation of optimized parameter settings, such as by requiring, in certain examples, additional programming instructions to proceed from the initial state.

In certain examples, the time period for determination and evaluation of changes can include a set amount of time (e.g., from minutes or hours up to a day, etc.) to ensure improved patient or ambulatory medical device condition, or a changing amount of time responsive to a relative difference in determined changes between programming states.

For example, if a component of the system simulates and predicts or receives an expected change, such as from a clinician or as an output of one or more machine learning models, and the received information of the current iterative programming state or a relative change associated therewith confirms such prediction, one or both of the time period for determination or the amount of variance between successive iterations can be extended or increased, whereas if the received information does not confirm such prediction, one or both of the time period for determination or the amount of variance between successive iterations can be reduced or in certain cases suspended or reversed, for example, back to a previous iteration or to the initial programming state, in certain examples with a notification of such suspension or reversion to the clinician, etc.

In certain examples, the system can apply parts of a programming change rather than the whole change at once. For example, if a programming change includes changes to multiple parameters, such as sensing and stimulation parameters, the multiple parameters can be adjusted in series rather than simultaneously. This approach helps identify the cause of any issues that may arise, as opposed to implementing all changes at once, which could make it difficult to determine which specific change elicited an adverse response.

For example, the programming instruction can include changes to both sensing and pacing parameters. In such example, the sensing parameters can be adjusted, incrementally or at once, prior to determining and administering incremental adjustments to the pacing parameters, or parameters that impact therapy, stimulation, or other intervention to the patient, to optimize sensing to verify the understanding of the clinician or system providing such programming instruction prior to adjusting pacing parameters that will impact the patient. Sensing parameters, in certain examples, refer to parameter settings of an ambulatory medical device that impact how information is sensed, detected, interpreted, or processed and can include, among other things, sensitivity settings, sensing thresholds, sensing vectors or electrodes, blanking periods, etc. Pacing parameters, on the other hand, refer to parameter settings of an ambulatory medical device that impact how energy or intervention is delivered or provided to the patient and can include, among other things, stimulation amplitudes, stimulation vectors, stimulation modes, rate limits, delay settings between or before stimulation or intervention, etc.

In certain examples, incremental adjustments to parameter settings and selectively applying changes to specific parameter sets can allow for continuous evaluation of patient response and device performance, which enables precise optimization of parameter settings while prioritizing patient safety and extending device longevity. Leveraging sophisticated digital models of both the patient and the device to support these change techniques, allowing for personalized and adaptive parameter optimization. These advancements in ambulatory medical device programming offer the potential for improved patient outcomes, more efficient use of device resources, and extended usable lifetimes of these critical medical devices.

Leveraging a comprehensive understanding of parameter relationships, such as described herein, can ensure that partial changes are applied in a manner that maintains the overall integrity and effectiveness of the programming, providing a powerful tool for fine-tuning device parameters while prioritizing patient safety and optimizing device performance. The changes over time and the separation of sensing and pacing parameters represent significant advancements in ambulatory medical device programming that allow for more nuanced and responsive adjustments to parameter settings for more efficient use of ambulatory medical device resources that can extend the usable lifetime of the ambulatory medical device while ensuring optimal therapy tailored to the evolving needs of the patient.

### Time Based Adjudication

Programming instructions can be communicated to the ambulatory medical device. The time between an instructed programming change and implementation of the programming change can vary in different programming environments, such as with patient proximity to a remote patient management device, etc. If the time period is too long, such that it exceeds one or more thresholds, the programming instruction can be stale. In such case, the programming instruction can expire (e.g., removed or held by the remote patient management system, the ambulatory medical device, etc.), such as after a threshold time without successful connection with, communication to, or received confirmation from the ambulatory medical device, for example, such as due to connection issues between the medical device programmer and the ambulatory medical device or a continued lack of proximity between the patient and the medical device programmer (e.g., more than 1 day, several days, etc.), increasing uncertainty of the current status of the patient and the ambulatory medical device.

In other examples, while a patient condition prior to a programming instruction was likely considered by a clinician prior to implementing the programming instruction, patient condition can change at or around the time of providing the programming instruction, or additional information can be received that indicates the patient condition considered by the clinician has changed at the time of the programming instruction. In other examples, the ambulatory medical device condition can change, such as a change in device orientation, loss of contact, sensing, or capture by one or more electrodes, a change in device parameters, state, or status, such as an estimated battery life remaining or a change in a rate of one or more parameters, etc., between the decision to change programming instructions and implementation. Accordingly, one or more changes can be detected (e.g., such as by the remote patient management system, the ambulatory medical device, etc.) and such detected changes can trigger expiration or suspension of the programming instruction, or in certain examples alert, notification, or additional confirmation by the clinician, etc., prior to implementation of the programming instructions. Such notification or alert can prompt a review of patient or ambulatory medical device condition, such as by prediction by one or more models or clinician review, etc.

Additionally, in certain examples, multiple programming instructions can be provided where a subsequent programming instruction is received by the ambulatory medical device prior to receipt or implementation of a previous programming instruction or a previous programming instruction could have been provided and in certain examples even received by the ambulatory medical device but not implemented prior to receiving the subsequent programming instruction. In such cases, it can be advantageous to suspend an instructed programming change and provide notification or alert of the multiple changes until a clinician reviews and validates the instructions, such as to avoid programming changes that do not take into account a full picture of the patient or the ambulatory medical device, or to ensure the patient or the ambulatory medical device does not implement a programming instruction and operate for a period with suboptimal programming, potentially reducing the useful lifetime of the ambulatory medical device and benefit of optimal programming to the patient.

Time-based adjudication of remote programming instructions can optimize programming of the ambulatory medical device, such as to optimize resources of the ambulatory medical device, reduce periods of suboptimal programming, and increasing the useful lifetime of the ambulatory medical device, the usefulness of the medical device to the patient and benefit of optimal programming to the patient, improving the safety and efficacy of remote device management, extending the period of utility provided to the patient prior to required replacement of the ambulatory medical device, etc. In certain examples, the programming instructions can include a time of the received programming instruction, such as a time received from a clinician or provided to the programmer, the ambulatory medical device, a desired time of implementation, etc., such as in the data of the programming instruction, as a field in the programming instruction, embedded in the instruction, as an object, string, data structure, in a file name, etc. Such features can ensure that programming instructions are applied in a timely manner and prevent application of outdated or potentially conflicting instructions. The time or percent change thresholds to trigger expiration or suspension can include a percent change from a baseline, such as an average time to implement a requested programming instruction, an expected change from the baseline, or one or more other thresholds, such as received by a clinician, etc.

### Implant and Follow-Up

When receiving a new medical device, patients may need to try several sets of parameter settings to receive sufficient or optimal therapy. In addition, in-clinic follow-up appointments are currently required as patient condition can change or response to existing devices or therapy can change over time, requiring additional changes to parameter settings that at one time were sufficient or optimal. In typical operation, a medical device, such as a cardiac resynchronization therapy device, is first programmed at a time of implant to a first operating mode, such as with respect to a first set of parameter settings, and then adjusted during scheduled in-clinic follow-up procedures with a clinician. Initial follow-up after implant (e.g., post-implant) or programming changes is generally a first time period, such as 4-6 weeks to allow patient recovery from the implant procedure and determination of baseline measurements for the patient from which to base future operation and monitoring, as well as to compare patient condition after implant or programming changes to the patient condition pre-implant or previous to programming changes. Subsequent follow-up after the initial follow-up can be less frequent, occurring, for example, every 3-6 months (e.g., at 6 months post-implant, at 12-months post-implant, etc.), or more or less as needed (e.g., between 1 and 12 months, etc.) depending on programming changes or changes in patient status or condition (e.g., a patient response metric). However, traditional follow-up appointments are in-person in a clinical setting and require travel for the patient, often at a substantial burden. In addition, programming changes may require additional follow-up, such as a new initial follow-up appointment and observation time, substantially increasing resources associated with programming the medical device and reducing the usable lifespan of the medical device during which the device is using limited resources to provide sufficient or optimal therapy.

### Patient Response to Parameter Settings

In certain examples, multiple therapies or combinations of parameter settings can be evaluated for efficacy for a particular patient using physiologic information from the particular patient without requiring in-person appointments, including implementing parameter setting changes (e.g., including changes in modes) from a medical device system including a remote component separate from the ambulatory medical device. In an example, instead of each change in parameter settings requiring an in-person clinic visit or follow-up appointment, a physiologic status of the patient can be remotely monitored through the ambulatory medical device to determine patient status (e.g., a patient response metric) in response to an implemented set of parameter settings, such as a series of different sets of parameter settings received or authorized at a time of implant or initial programming.

For example, the systems and methods can be configured to determine a value or trend indicative of patient status in response to a first set of parameter settings (e.g., determining if the first set of parameter settings are beneficial to the patient or if a change in parameter settings are needed). If the determined patient status to the first set of parameter settings is below a threshold (e.g., an expected patient status or a relative threshold determined as a function of a previous patient status, etc.), a second set of parameter settings can be implemented remotely by the medical device system through the ambulatory medical device. Like with respect to the first set of parameter settings, the systems and methods can be configured to determine patient status in response to updated or changed parameter settings (e.g., the second set of parameter settings, etc.) to determine if the updated or changed parameter settings are beneficial to the patient or if additional changes in parameter settings or additional follow-up is needed. In certain examples, the determined patient status can be used to trigger an alert to a clinician or an adjustment to a follow-up schedule, such as if a value of the determined patient status is above or below one or more thresholds associated with an alert or follow-up requirement, etc.

As therapy progresses, physiologic characteristics of the heart change. Accordingly, even after an initial therapy has been optimized, it is important to periodically reassess and re-optimize therapy and parameter settings at extended intervals (e.g., yearly, etc.). Accordingly, even if patient status is above a healthy threshold, one or more parameter settings or modes can be adjusted to determine positive or negative patient response to the adjustments, such as to guide additional programming changes to continue to provide optimal therapy or parameter settings.

In addition, an automated “operation system test” or “system self-evaluation” on device function or parameter settings can confirm continued optimal operation. In an example, one or more parameter settings can be changed or adjusted and one or more indications of patient condition (e.g., patient response metrics, indications of patient status, risk indications, etc.) can be determined in response thereto. In an example, if the indication of patient condition falls or deteriorates in response to the change or adjustment, device function or parameter settings can be programmed to revert to the previous operation. In other examples, one or more other changes or adjustments can be programmed, in contrast to such changes that provided the previous deterioration in patient condition, and a responsive indication of patient condition can be determined to guide additional changes, adjustments, or reversion to previous operation.

In certain examples, a clinician or a user can implement a stop action to revert or rollback a change in parameter settings in response to negative changes in patient response metrics (e.g., rates, thresholds, impedance, adverse medical events, etc.) after a change. Such stop action can be received by one or more circuits, for example, through an external or a remote device, and can include an open-ended stop action to revert to a previous operation or a time-based stop action allowing a user to provide a stop action to be implemented for a specific post-change time period (e.g., several hours, up to a day, etc.). In other examples, patient response metrics after a change in parameter settings can be evaluated by an external or remote device to recommend or implement a stop action or provide one or more alerts, etc., in response to negative or adverse findings.

Advantages of the systems and methods described herein include, among other things, optimized power usage by the ambulatory medical device, extending a usable lifespan of the limited power resources (e.g., battery) by more quickly optimizing therapy through successive changes in parameter settings, additionally include optimization of clinician resources and follow-up scheduling, as well as reducing the need for patient self-reporting of status following a change in parameter settings, allowing a more responsive and objective reporting and management system.

Implant and follow-up schedules for an ambulatory medical device can vary significantly among patients, devices, or clinicians from time (0) at a time of implant through a first time period (e.g., 1 year) of evaluation and operation. A traditional initial follow-up (in-clinic) can occur at 1 month (e.g., 4 weeks) after implant with subsequent follow-ups (in-clinic) at 6 months and 1 year. Subsequent follow-ups can continue after 1 year at this (e.g., every 6 months) or other durations. Changes to parameter settings can be determined and programmed to the ambulatory medical device by a clinician during follow-up appointments, with additional initial follow-up (e.g., 1 month after a parameter setting change) following each change in parameter settings.

Remote reporting can reduce in-clinic follow-up requirements. For example, a report can be determined, such as by a patient management system, and provided to a clinician at a first time period (e.g., including a recovery period and time to determine a baseline response, such as 1 month, etc.) following implant or a change in programming or parameter settings. In this example, the report can be provided remotely, without an in-clinic follow-up appointment, unless required by a clinician, etc. In response to the report, the clinician can program changes to the parameter settings, such as through the patient management system, to be implemented by the ambulatory medical device without an in-clinic follow-up, or alternatively hold existing settings or schedule an in-clinic follow-up if necessary or desired. After a second time period (e.g., a subsequent recovery period, such as an additional 1 month, etc.) after the change in parameter settings, an additional report can be determined and provided to the clinician. In an example, the report can include a determined patient response metric or patient status or condition based upon received physiologic information for the respective time period, or a comparison of the determined patient response metric for the respective time period in contrast to a determined patient response metric for a previous time period, such as a time before implant, or a time before a prior change, etc. In an example, if no subsequent changes are provided or desired by the clinician, traditional in-clinic follow-up can continue 1 year or one or more other time periods or durations. Also, as noted above, it can be important to re-optimize at longer time periods (e.g., such as at 1 year or greater). In certain examples, it can be beneficial to perform re-optimization in the months or weeks prior to a scheduled in-clinic follow-up, such that the changes are before but close in time to a planned in-clinic visit, reducing subsequent follow-up appointments.

In certain examples, changes can be determined automatically by a patient management system, such as through one or more automated systems configured to analyze parameter settings and generate reprogramming recommendations for ambulatory medical devices to optimize or improve patient condition. In one example, such as with respect to cardiac resynchronization therapy patients, patient condition can be determined with respect to a percentage of confirmed cardiac capture resulting from pacing stimulation, etc. In certain examples, a clinician, at the time of implant or after a recovery period, can authorize a range of settings or a series of settings for the device to cycle and optimize through successive changes and analysis, remotely (e.g., remote from the patient, such as through a cloud-based connection), without in-clinic requirements for each change in parameter settings. If responses are within acceptable guidelines or normal expected values at respective recovery periods (e.g., 1 month after a change, etc.), reports can be placed in the patient medical records without an alert to the clinician until the range or series of settings have been implemented and evaluated, or until a determined patient response exceeds a threshold.

For example, after a recovery period (e.g., 1 month after implant), a first change can include a change from the recovery or monitoring period to the first therapy, or a change from the first therapy to a second therapy. A second change after a second period (e.g., at 2 months) can include a subsequent change different than the first change. A third change (e.g., at 3 months) can include a subsequent change different than the first or second changes, or in certain examples, if one of the previous therapy or sets of parameter settings provided a better patient response metric (higher if a higher value of the patient response metric indicates a positive patient condition, lower if a higher value of the patient response metric indicates a worsening patient condition), the third change can include reverting back to the therapy having the better patient response metric (e.g., from a second therapy to a first therapy, etc.), and away from the therapy or time period indicating a worse patient status.

Although discussed herein as specific time periods, such as pre-determined time periods, etc., in other examples, the rate of change, follow-up, report, recovery, or implementation of initial or subsequent programming or therapy can include one or more other time periods determined by a clinician, determined by patient response metrics or physiologic information of the patient, or one or more other time periods. In an example, a lock-out period can be implemented by one or more devices or circuits or set by a clinician to prohibit device changes in modes or parameter settings in the lock-out period (e.g., one week, two weeks, etc.) prior to a scheduled follow-up appointment, such as to avoid a changing patient condition during subsequent follow-up which may lead to additional unnecessary device changes, enforcing a lock-out period on the device side. In other examples, a follow-up schedule can be modified based on a time of most recent change in parameter settings, such as to avoid a changing patient condition during subsequent follow-up which may lead to additional unnecessary device changes, enforcing the lock-out period on the clinician side.

In an example, the patient management system can be configured to analyze different pacing parameters using artificial intelligence or machine learning, based on received physiologic information or separate therefrom, to identify optimal and suboptimal combinations of pacing parameters corresponding to one or more conditions, such as to optimize patient condition, to improve rates of cardiac capture resulting from pacing stimulation, to eliminate or reduce periods of suboptimal, missed, or reduced pacing, etc. Data can be collected and organized for analysis and identification of patterns. Models can be created based on the identified patterns, validated (e.g., using a percentage of confirmed LV cardiac capture, etc.), stored, and deployed. Additionally, deployed models can be monitored and updated as additional data is collected, including retraining as needed.

Medical device systems can analyze physiologic information between patients or with respect to one or more clinical thresholds to determine patient condition and optimize device settings. In other examples, analysis can focus on differences between the parameter settings themselves (e.g., without respect to patient physiologic information, determined indications of cardiac capture or reduced pacing, patient demographics, patient history, etc.), such that a determined similarity between different parameter settings for the same or different patients can be analyzed to identify sub-optimal settings or combinations of settings that may result in suboptimal, missed, or reduced pacing. In certain examples, parameter settings can be additionally analyzed with respect to one or more of patient physiologic information (e.g., to identify similar patients, etc.), determined indications of cardiac capture or reduced pacing, patient demographics, patient history, or combinations thereof.

Clinicians have broad discretion in determining and implementing parameter settings of medical devices (e.g., CRT devices, etc.) but often follow published literature and guidelines or specific device limits. However, as recommendations change or new therapies, modes, parameters, or settings are introduced, it takes time for such literature or changes in such literature to become widely understood and adopted. For example, certain clinicians may have determined a specific set of parameter settings to optimize pacing in certain patient populations that differ from the previous literature or clinician training. Analysis of settings on a between-patient or between-clinician basis with respect to optimized pacing or capture can identify and determine different combinations of settings and distribute recommended sets of parameter settings more quickly than existing literature. Additionally, whereas clinicians focus on certain parameters, with access to a complete set of parameter settings across large numbers of patients, correlation between seemingly irrelevant parameters in combination with others can be determined that impact cardiac capture rates, improving pacing and cardiac resynchronization therapy, patient outcomes, device performance and efficiency, and communication of leading clinical data more quickly to clinician populations.

In an example, a desired clinical outcome can be provided to the patient management system, such as to optimize cardiac capture. Once the desired clinical outcome is received, changes to therapies or parameter settings can be determined automatically by the patient management system, such as through one or more automated systems configured to analyze parameter settings and generate reprogramming recommendations for ambulatory medical devices to achieve the desired clinical outcome, for example, by optimizing parameter settings to improve a percentage of confirmed cardiac capture resulting from pacing stimulation, etc.

### Machine Learning and Artificial Intelligence

Artificial intelligence, particularly machine learning and other techniques, can effectuate the speed and analysis of identifying optimal settings and determining differences between different sets of parameter settings, in combination with physiologic information of the patient (such as determination of patient status, e.g., improving or worsening, etc.) or separate therefrom, taking into account rates of cardiac capture in specific patients or across populations. In addition, separate from tracking rates of cardiac capture for specific patients or patients having specific demographics, disease states, or patient conditions, rates for specific clinicians can be analyzed and determined to identify clinicians having more successful rates of cardiac capture across patients or patient groups.

For example, based on a specific desired output, such as optimizing cardiac resynchronization therapy by effectuating cardiac capture, etc., pacing parameter settings can be analyzed to identify or determine specific parameters or combinations thereof that are more likely correspond to unconfirmed or missed cardiac capture. In an example, although parameter settings often start from a default condition and are separately selectable and adjustable by a clinician, combinations of parameters often ideally move together. In an example, if one parameter is adjusted and a second is not, but adjustment of the second often provides optimal cardiac resynchronization therapy, detection of the second not being adjusted can trigger a recommendation to the clinician to adjust the second parameter. In other examples, such parameters can be adjusted automatically by the system with notice provided to the clinician of such change for review or approval.

In other examples, such as first programming after implant, or situations where patient physiologic information has not been previously recorded or is otherwise unavailable, proposed parameter settings can be recommended based on other information about the patient, such as age, gender, medications, co-morbidities, diagnosed conditions or disease states or progressions, or other information medical history information separate from sensed physiologic information. In this way, the first programmed values for a specific patient can differ from default values for all patients, potentially improving the speed of attaining optimal programming and reducing wasted resources associated with suboptimal operation.

Additionally, optimal parameter settings, or suggested combinations of parameter settings to optimize cardiac resynchronization therapy, may adjust over time, just as the parameters and settings themselves. In certain examples, optimal combinations of parameter settings or suggestions to optimize parameters settings for a particular patient can be provided to a clinician, such as during follow-up or report. In other examples, suboptimal pacing, including unconfirmed or confirmed missed cardiac capture, or a determined patient status or condition lower than a threshold or an expected improvement can trigger analysis and recommendation.

Periods of suboptimal therapy can be harmful to patients but may also lead to inefficient use of device resources including periods of stimulation by the device that may not provide a desired physiologic response, effectively wasting limited device resources. Identifying potentially less effective or ineffective parameter settings or combinations of parameter settings and providing a recommendation of one or more programming changes to improve pacing can result in a more efficient use of device resources while also improving patient therapy. Accordingly, identification of suboptimal settings and generating reprogramming recommendations can improve operation of the underlying hardware.

Parameter settings can be tracked, including patterns of changes across different patients and resulting impact, for example, on patient condition, cardiac capture, etc. Capture can include confirmed capture of one or more chambers, such as confirmed LV cardiac capture, confirmed RV cardiac capture, confirmed RA cardiac capture, or combinations thereof (e.g., confirmed Bi-V cardiac capture, including RV and LV, confirmed LV-only, etc.). In an example, confirmed cardiac capture in less than 98% of cardiac beats (or in other examples, less than 95% or less than 90%) over a period of time, such as a week, a day, a group of successive beats, etc., can trigger an alert or notification and analysis or re-analysis of device parameter settings. In other examples, a reduced trend of confirmed cardiac capture over time, or a sudden loss of cardiac capture below a threshold (e.g., from above 98% to lower than 90%, 80%, 50%, 20%, 10%, or to 0%, etc.) can trigger an alert or notification and analysis or re-analysis of device parameter settings. In other examples, all sets of parameter settings can be analyzed with respect to model parameter settings to identify suboptimal programming and suggest changes, in certain examples, additionally with respect to confirmed cardiac capture percentage. For example, if key opinion leaders change a model set of parameter settings, even in situations where a patient has confirmed cardiac capture at 98% or above, a notification can be provided to a clinician illustrating the differences and impact of such to the medical device and the patient.

In an example, the one or more pre-trained machine learning models can be trained, in certain examples, such as described herein. For example, the determined patient response metric and parameter settings can be input into one or more pre-trained machine learning models trained to compare the received parameter settings to stored model parameter settings from one or more other implantable medical devices corresponding to one or more other patients and to identify one or more differences between the parameter settings of the implantable medical device and the stored model parameter settings of the one or more other implantable medical devices and different determined patient response metrics. Upon obtaining an output from the one or more pre-trained machine learning models indicating the identified one or more differences between the parameter settings of the implantable medical device and parameter settings of the one or more other implantable medical devices, a programming recommendation can be generated for the implantable medical device to improve patient condition (e.g., cardiac capture) for the patient based on the identified one or more differences, or device performance (e.g., resource usage) without negatively impacting patient condition, etc.

In other examples, if the determined patient response metric indicates that the existing therapy is stable or changing, etc., one or more modes or functions of the implantable or ambulatory medical device can be altered to increase or decrease a power consumption or sensing or storage capability of the implantable or ambulatory medical device. For example, one or more hardware limitations can be adjusted, such as to, among others: sense or receive more or less physiologic information of the patient; increase or decrease communication frequency between the implantable or ambulatory medical device and an external device (e.g., remote device, programmer, etc.), such as to increase or reduce the frequency of patient monitoring, etc. ; switch to a different power or resource intensive monitoring algorithm; etc. In an example, if the determined patient response metric is stable after an extended period of time (e.g., greater than 6 months, greater than one year, greater than two years, five years, etc.), programming recommendations can be provided to a clinician for review, or implemented automatically, to reduce power consumption or extend device lifespan without negatively impacting the patient. In other examples, if the determined patient response metric has been stable for an extended period of time but then starts to drift (e.g., starts to slowly decrease, but not yet triggering any alert thresholds, etc.), one or more modes or parameter settings can be adjusted to determine if different parameter settings can reduce drift or improve patient condition before a subsequent follow-up. In certain examples, changes can be prohibited if the determined patient response metric is not stable at a time of a proposed change.

In addition, during programming or reprogramming of medical devices, related parameter settings can be identified and grouped, such as to suggest corresponding changes (e.g., during initial programming, during follow-up sessions, etc.), where one parameter setting is changed, suggesting corresponding changes to one or more other programmer settings to optimize therapy to the patient. In addition, values of such parameter settings can be analyzed to suggest not only specific parameter settings to program or reprogram, but values of different parameters based upon proposed changes. For example, if changing one parameter by a first amount, a recommendation can be provided to change a second parameter by a second amount. In contrast, if changing the one parameter by an amount greater than the first amount, the recommendation can be changed to change the second or one or more other parameters by an among different than the second amount, greater or less, depending on the specific parameters.

A recommendation system, such as comprising one or more assessment circuits, can utilize artificial intelligence and machine learning models to identify corresponding changes, for example, corresponding to positive outcomes (or away from negative outcomes), such as with respect to patient condition, confirmed cardiac capture percentages of cardiac beats, or one or more other desired clinical outcomes in other patients. In certain examples, parameter settings by key opinion leaders can be weighed more heavily than parameter settings by other clinicians. In other examples, specific models based on parameter settings of key opinion leaders, including individual clinicians or groups of clinicians at the forefront of thought leadership in the field of cardiac rhythm management therapy, can be suggested, separate from other clinicians. Combinations of parameter settings (e.g., including patterns, values, etc.) can be identified and validated to optimize patient condition, cardiac capture, etc., and validated identified parameter settings can be recommended as optimal values to clinicians, such as when programming or reprogramming medical devices, including implantable medical devices, etc. In certain examples, validation can include combinations of human and machine validation confirmed based on stored information and recorded patient outcomes.

In other examples, other potential suboptimal pacing scenarios can be improved using the systems and methods described herein, such as identifying suboptimal parameter settings associated with high pacing (e.g., a percentage of paced beats above a high or desired pacing threshold) in an implantable cardioverter defibrillator (ICD) (e.g., not a cardiac rhythm management device, but a defibrillator), or chronic overpacing in one or more ambulatory medical devices, where a patient condition could have changed such that existing settings, although resulting in cardiac capture, are not required any longer. For example, patient physiologic information, such as heart sound information (e.g., S1 amplitude, providing an indication of cardiac contractility of a ventricle, etc.) can be used to triage patients and determine whether periods of intrinsic activity should be allowed, or if one or more parameter settings should be adjusted or recommended commensurate with a change in the patient physiologic information or patient status determined using such information.

### Digital Models

Outcomes of programming changes for patients and ambulatory medical devices can be predicted, or in certain examples programming recommendations themselves can be made, using one or more digital models, such as a digital model of a patient, a digital model of an ambulatory medical device, or combinations thereof, to optimize resources of the ambulatory medical device, thereby extending a usable lifetime of the ambulatory medical device, such as with respect to utility provided to the patient prior to required replacement of the ambulatory medical device, etc. The one or more digital models (e.g., cloud-based digital models accessed, generated, or updated by the systems and methods described herein) can include a digital model of a particular patient, a digital model for a particular ambulatory medical device or particular type or configuration of a particular ambulatory medical device, or in certain examples combinations thereof. In this way, evaluation, prediction, and recommendations can be patient-centric (e.g., based first on a generated digital model of the patient), device-centric (e.g., based first on a generated digital model of an ambulatory medical device), or in other examples patient-and-device-centric (e.g., combinations of both, first one then the other, etc.).

In an example, the digital model of the patient can be generated for a particular patient based on physiologic information received from an ambulatory medical device implanted in, located on, or otherwise associated with the particular patient and be continuously updated as additional physiologic information is received, such as from the ambulatory medical device, from one or more other ambulatory medical devices associated with the particular patient, or in certain examples from determined indications, comorbidities, disease states, detected events, or information from medical records of the particular patient or received from the particular patient, such as patient demographics, etc.

In an example, the digital model of the ambulatory medical device can be generated for a respective ambulatory medical device based on information about the ambulatory medical device, such as based on a type, configuration, parameter settings, or in certain examples further with respect to physiologic information of one or more other patients having the type, configuration, or parameter settings of ambulatory medical device, etc.

Each of the one or more digital models has distinct advantages. For example, the digital model of the patient (e.g., a patient-centric model) can predict which of a plurality of different types of ambulatory medical device or configuration or parameter settings might provide the most benefit to the patient or a respective ambulatory medical device based on physiologic information from one or more other patients having similar physiologic information to the particular patient. In contrast, the digital model of the ambulatory medical device (e.g., a device-centric model) can predict which parameter setting or set of parameter settings might provide the most benefit to the particular patient or a respective ambulatory medical device given the type, configuration, or parameter settings of ambulatory medical device, resulting in a smaller amount of required computing resources (a smaller training set of data) or improved outcomes in situations where physiologic information from one or more other patients having similar physiologic information to the particular patient is lacking in the training data.

Additionally, concentrating on ambulatory medical device performance, for example, expected performance versus observed performance according to the digital model of the ambulatory medical device, enables monitoring and control of ambulatory device behavior and condition in a more focused and efficient way than through patient monitoring. Whereas device performance can be one aspect that results in changes to changes in a digital model of a patient, certain patients, in periods of good health, may tolerate or compensate for poor ambulatory medical device performance, but if patient condition worsens, the poor ambulatory medical device performance can accelerate patient decline in a time period where the patient may not be healthy enough to change the ambulatory medical device. Separately, concentrating on patient performance, for example, expected patient performance versus observed performance according to the digital model of the patient, maintains patient status and condition as a hallmark, separate from ambulatory medical device performance. Analyzing programming changes by simulating and predicting outcomes on the one or more digital models and comparing post-programming outcomes enables focused across-device or across-patient analysis at the areas where the machine learning model or training data is weakest, while also identifying beneficial programming changes across different patients or devices.

In an example, when a proposed programming change is received, such as a therapy modification or changes to parameter settings, the systems and methods can simulate application of the change on the one or more digital models to determine impact on the digital model of the patient or the digital model of the ambulatory medical device (or in certain examples combinations thereof), such as to predict or analyze the impact of the proposed programming change on the patient or the ambulatory medical device. By leveraging training data from a broad set of patients and devices, the systems and methods can predict the likely outcome of the proposed change and generate a recommendation regarding implementation, in certain examples confirming or denying a proposed programming change or in other examples generating one or more alternative programming changes providing more positive impact on the digital model of the patient, the digital model of the ambulatory medical device, or combinations thereof. Evaluating proposed programming changes by simulation on digital models improves safety and efficacy of device management while additionally sharing educational advances in programming and device management of physiologic conditions, further improving safety and efficacy of existing devices, reducing the likelihood of negative programming impacts to patients and resources associated with suboptimal parameter settings or device choices, and optimize resources of the ambulatory medical device.

A broad set of patient data and detected events can be leveraged to evaluate different types of programming changes for ambulatory medical devices, in certain examples in real-time and continuously after implementation. By simulating potential changes using the digital models before actually implementing them, the systems and methods can provide a comprehensive evaluation of the likely success of a programming change. This evaluation may include a percent confidence, a risk assessment (e.g., High/Medium/Low risk), and indications of potential concerns, issues, or edge cases that may arise.

The digital model of the ambulatory medical device can be created with a large amount of physiologic information from different input signals, such as cardiac electrical information, heart sounds, respiration, impedance pressure, activity, or other physiologic information or parameters of the patient. This large amount of physiologic information allows the system to evaluate simulated performance and produce more accurate outcome predictions based on previous implementation in similar situations, or implementations on either side of the specific proposed programming changes. For example, the systems and methods can simulate the effects of switching sensing vectors or changing sensing parameters based on device programming data.

In certain examples, the systems and methods can provide guidance on what changes should be made to achieve a desired clinical outcome. Clinicians can input clinical goals for the particular patient and the digital models, trained on a vast array of patient data and outcomes, can suggest specific programming changes to accomplish those goals. This “reverse engineering” approach allows clinicians to focus on the desired patient outcome rather than the technical details of device programming while also leveraging state-of-the-art literature and guidelines that may not yet be recognized by the clinician.

As the systems and methods described herein continues to receive and analyze data from a growing patient population, its predictive capabilities and recommendations will become increasingly refined and accurate. The combination of continuously updated patient models, device simulations, and AI-driven recommendations has the potential to significantly improve the efficiency and effectiveness of implantable medical device management, ultimately leading to better patient outcomes and use of device resources, increasing effectiveness of devices and increasing device lifetime and the percentage of optimal impact on the patient over the limited lifespan of the device.

A cloud-based system, or a system having a cloud-based component, for predicting and evaluating programming changes to implantable medical devices offers several key advantages over traditional programming methods. For example, by leveraging vast amounts of patient data and sophisticated digital models, the system can provide more accurate and personalized recommendations for device optimization that provide substantive benefits to the device and patients associated therewith.

For example, the systems and methods described herein can create and maintain a digital “twin” of the particular patient (e.g., the digital model of the patient) and associated ambulatory medical device (e.g., the digital model of the ambulatory medical device). Such digital twins can include highly detailed virtual representation of the physiologic information of the particular patient and the associated ambulatory medical device, incorporating not only the device specifications and current settings but also simulated input signals that can mimic actual patient physiological data. By continuously updating these digital twins with real-time data from the patient and patient device, the systems and methods described herein can maintain an accurate model for simulation and prediction purposes, but also for interrogation by clinicians for reporting, education, or compliance, similar to a black box in an airplane.

In certain examples, the predictive capabilities can extend beyond simple parameter adjustments. By analyzing patterns in large datasets from multiple patients, the digital models can identify complex relationships between device settings, patient characteristics, and clinical outcomes. This allows the systems and methods described herein to suggest novel programming strategies that may not be immediately apparent to human clinicians, potentially leading to improved patient outcomes in challenging cases.

Further, as the patient model increases in complexity and nuance, less physiologic information may need to be communicated outside of an implantable medical device if the information is not adding to or deviating from the existing patient model—redundant information may not need to be recorded, stored, reported, or communicated out of the device using one or more communication circuits, which is commonly the largest source of power consumption in the ambulatory medica device, directly impacting device lifespan. In one example, storage or communication of physiologic data outside of an ambulatory or implantable medical device can be omitted if the physiologic information agrees with expected values. Moreover, storage and communication by the ambulatory medical device can be controlled by different parameter settings of the ambulatory medical device associated with each. For example, determinations, storage, recordings of high-resolution physiologic data (e.g., electrogram or HS segments, etc.), or transmission of physiologic information (e.g., at a higher resolution or sampling frequency, such as by a loop recorder, etc.) can be omitted, such as by different parameter settings, if physiologic parameters are within expected values of the digital model. In other examples, ambulatory medical device resources can be adjusted to improve efficiency, such as by reducing sampling frequency of one or more sensors, reduce data processing, storage, or transmission of information outside of the ambulatory medical device, optimizing resources of the ambulatory medical device. Once the physiologic information deviates from the digital model, such as by more than a deviation threshold, storage and transmission can be triggered, such as by adjusting one or more parameter settings, thereby optimizing device resources. In certain examples, the additional data from the patient or the ambulatory medical device that disagrees with the existing training data may need to be processed into the training data, updating the machine learning model. However, subsequent updates of the training data will lead to more efficient subsequent training of the ambulatory medical device, optimizing resources of the ambulatory medical device, as well as optimizing resources of other ambulatory medical devices for the patient or other patients.

The digital models described herein are designed to be continuously learning and improving. As more data is collected and more programming changes are evaluated, the predictive accuracy and the relevance of recommendations will increase. This creates a positive feedback loop, where better recommendations lead to improved patient outcomes, which in turn provide more valuable data for further refinement of the models.

In addition, “what-if” analyses can be performed using the one or more digital models with respect to the patient, the ambulatory medical device, or one or more other patients or ambulatory medical devices. Through interaction with the different models, clinicians can propose and explore multiple potential programming changes simultaneously, comparing predicted outcomes and risk assessments for each scenario. This allows for a more comprehensive evaluation of treatment options and can help clinicians make more informed decisions about device management and in certain examples the impact to the particular patient of different parameter settings, therapies, or even different ambulatory medical devices, which can be especially beneficial in determining which type of device to implant, which configuration to implement, or when to exchange or replace existing devices.

Cloud-based aspects of the system additionally facilitate collaboration and knowledge sharing among healthcare providers. Anonymized data and insights gained from successful programming strategies can be shared across institutions, potentially accelerating the adoption of best practices in device management and improving overall standards of care for patients with implantable medical devices.

The one or more digital models can serve as a valuable tool for patient education and engagement. Providing clear visualizations of predicted outcomes and explaining the rationale behind recommended changes can help patients better understand their treatment and potentially improve adherence to medical advice, improving device utilization and outcome and potentially reducing deliberation time by patients prior to implant, implementation, or adoption.

In certain examples, instead of generating the digital model of the patient or the ambulatory medical device, the systems and methods herein can access a stored digital model for use in evaluating different ambulatory medical devices or programming changes for ambulatory medical devices, such as by simulating different parameter settings for one or more ambulatory medical devices on the digital model of the patient and updating the digital model of the patient, the ambulatory medical device, or combinations thereof prior to selecting, programming, or reprogramming an ambulatory medical device.

### Feature Extraction

FIG. 4 illustrates an example 400 of a table 401 of parameter settings and a reduction 402 of different combinations of parameter settings. In an example, specific parameter settings for a medical device can be represented by changes or deviation from a mode (e.g., a most frequent, or in other examples another statistical parameter, such as an average, a median, etc.) for a respective parameter setting from other medical devices, other medical devices in similar patients (e.g., grouped by disease, physiologic information, patient status, or combinations thereof), or the same medical device in a patient but at a different times. For example, whereas each medical device has a set of parameter settings, respective medical devices also have different sets of parameter settings at different times.

A kernel is a type of function used in various AI and machine learning algorithms that enables an algorithm to operate in high-dimension space without computing the coordinates of the data in that space. For example, a kernel can compute a dot product of multiple vectors without having to compute the coordinates of the points in that space, saving computational resources. In an example, a kernel can be used to extract and store patterns in different sets of parameter settings of cardiac resynchronization therapy or other medical devices (e.g., an implantable cardioverter defibrillator (ICD), etc.) and to identify rules for different sets of parameters for specific sets or classes of devices performing similar functions, in certain examples, further tailored to specific patients based on physiologic information of such patients.

In an example, parameter settings for one medical device are illustrated in the table 401 for example, across different office visits or sets of programming changes, etc. The changes in settings are illustrated with a 1 or a 0 in the table 401. For example, going from a 0 to a 1 can indicate that a previous setting was not utilized in the 0 state but is not utilized in the 1 state. In other examples, 0 and 1 can include a range of values that the parameter is outside of or a value that the parameter is above or below, etc., such as indicated with a 0, or a range of values that the parameter is now inside of or a value that the parameter is below or above, etc., such as indicated with a 1.

Columns or rows of the table 401, or in certain examples the table 401 itself, depending on organization, can be treated as vectors. In an example, columns A-D illustrate four different table entries. However, the four table entries A-X illustrate a number of parameter settings, P1-PN, with P1 having two states (P1A and P1B), reflecting different changes, for example, from a mode (most used value or setting for a particular parameter across a group of patients, such as in the training data, etc.) to a first value, illustrated as P1A, or from the mode to a second value, illustrated as P1B.

In a particular example, P1 can include an offset value, such as a sensed atrioventricular delay (AVD) offset value of 30, 40, or 50 ms, with a mode of 30 ms. P1A can represent a change from 30 ms to 40 ms, and P1B can represent a change from 30 ms to 50 ms. In other examples, additional table entries can be included, such as P1C illustrating a change from 40 ms to 50 ms, or other values, parameters, etc. P2 through PN can include other parameter settings, such as particular sensing or tracking modes with 0 representing the mode (e.g., on or off) and 1 representing a change from the mode (e.g., off or on, respectively). As with P1 including P1A and P1B, P2, . . . PN can also include other states as needed.

Parameter settings can be transformed into a table format or vectors, such as described herein (e.g., variance from a mode), and different combinations of settings and changes resulting therefrom (e.g., % of atrial or ventricular cardiac capture, etc.) can be evaluated to identify sets of parameter values or corresponding changes that represent the greatest positive impact, or positive impact above a threshold.

For example, changes in parameter settings can be evaluated to identify candidate rules or parameters values or combinations of parameter values that exceed a minimum support value (α) above a threshold (e.g., an upper percentile of response, etc.) to produce a reduction 402 of combinations of parameter settings. The minimum support value can be a function of frequency, impact, or combinations thereof.

In certain examples, upward closed analysis (or other analysis) can be used to reduce the overall work required to identify combinations of parameter values and associated rules. Upward closed analysis generally refers to a property where, if first and second parameter values are below a minimum support value (e.g., P(A)<α and P(B)<α), then any combination of parameters including the first and second parameter values will be below the minimum support value (P(A, B)<α). Based on such analysis, the reduction 402 illustrates that if P(X)<α, then all combinations including P(X) can be excluded from analysis. Similarly, if P(B, C)<α, then all combinations including P(B, C) can be excluded from analysis.

Although described herein with respect to upward closed analysis, in other examples other types of analysis can similarly be used, such as lower closed analysis or other order or lattice theory elements or properties, etc. Additionally, although discussed herein with respect to a single patient or a single medical device, in other examples, parameters can be evaluated across different patients, across different medical devices, or combinations thereof.

### Machine Learning Model

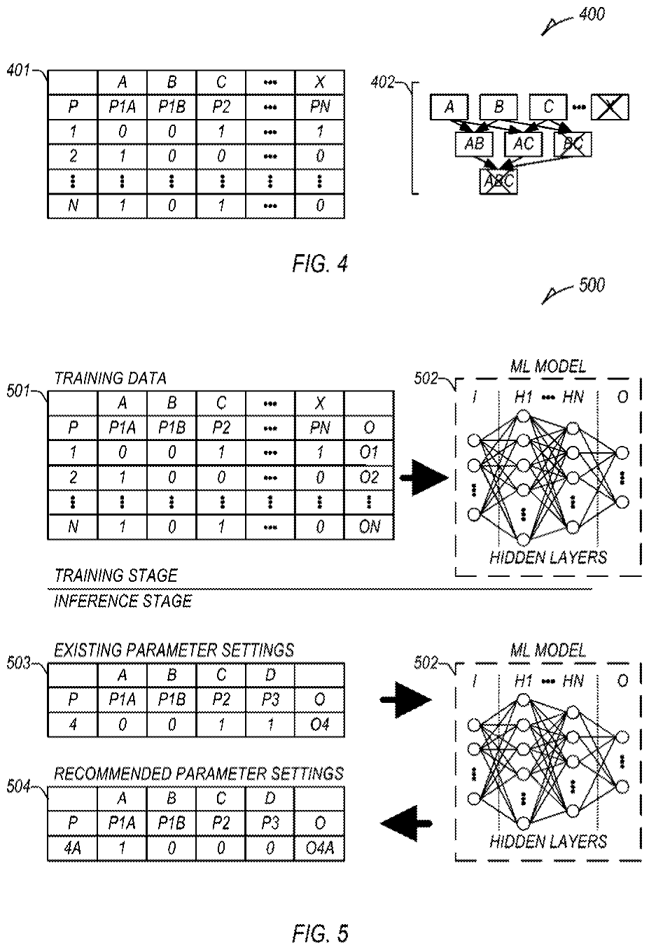

FIG. 5 illustrates a diagram 500 of an example machine learning model 502 trained to receive, as input, training data 501, such as parameter settings from one or more ambulatory medical devices, with respect to one or more patients, and in certain examples physiologic information associated with respective parameter settings, such as patient condition of the one or more patients, associated cardiac capture rates of different patients, or other physiologic information, etc., to identify optimal or suboptimal parameter settings or combinations of parameter settings corresponding to a desired clinical outcome, such as successful cardiac resynchronization therapy, patient status or condition, etc., and to generate an output indicating proposed parameter settings to optimize the desired clinical outcome (e.g., to optimize cardiac resynchronization therapy, such as to reduce suboptimal, missed, or reduced pacing based on existing parameter settings, to improve patient condition, etc.).

In the training (or learning) stage of FIG. 5, the training data 501 can be applied to a machine learning algorithm to train the machine learning model 502. In an example, the machine learning model 502 can include (1) a digital model of a patient, such as based on received physiologic information of a patient, (2) a digital model of an ambulatory medical device, such as based on one or more parameter settings or combinations of parameter settings, configuration, or type of the ambulatory medical device or a plurality of other ambulatory medical devices or in certain examples based on received physiologic information from one or a plurality of patients, or (3) combinations or permutations thereof.

The training data 501 can include parameter settings (e.g., P1A, P1B, P2, . . . PN, etc., such as described in FIG. 5) from a number of different medical devices (e.g., 1-N, etc.). Although illustrated as parameters settings (e.g., P1A, P1B, P2, . . . PN) and medical devices (1-N), in practice the training data 501 can include substantially more parameter settings and medical devices, such as tens or hundreds of parameter settings and hundreds or thousands of medical devices. In certain examples, the same medical device can provide parameter settings at different times. In certain examples, each vector or set of parameter settings can include an outcome (O) or assessment information, such as patient physiologic information (e.g., cardiac capture information, etc.) or other information of or about a patient associated with the respective parameter settings. In certain examples, the assessment can be provided by or validated by a user, such as based on direct measurement of patient physiologic information, etc.

The machine learning model 502 can be trained using supervised learning, unsupervised learning, or reinforcement learning. Examples of machine learning model architectures and algorithms may include, for example, decision trees, neural networks, support vector machines, or deep neural networks, etc. Examples of deep neural networks can include a convolutional neural network (CNN), a recurrent neural network (RNN), a deep belief network (DBN), a long-term and short-term memory (LSTM) network, a transfer learning network, or a hybrid neural network comprising two or more neural network models of different types or different model configurations. The training of the machine learning model may be performed continuously or periodically, or in near real time as additional patient data are made available. The training involves algorithmically adjusting one or more model parameters or parameter settings, until the model being trained satisfies a specified training convergence criterion. The machine learning model 502 can establish a correspondence between parameter settings or combinations of parameter settings and patient condition, cardiac capture, etc.

For example, with respect to an outcome, such as cardiac capture, a machine learning model can be trained to analyze physiological signal data and detect a respective outcome, such as cardiac capture or successful or optimal cardiac resynchronization therapy, or correspondingly, suboptimal cardiac resynchronization therapy. To train such a model, for example with respect to cardiac capture, a dataset is assembled containing sample patient physiologic information annotated by medical experts to identify successful cardiac capture and correspondingly, unconfirmed cardiac capture or unsuccessful cardiac capture. This annotated training dataset is then used to train the machine learning model 502 using a supervised learning approach, such as by algorithmically adjusting internal parameters to map the input patient physiologic information to the expert-applied labels. Various machine learning algorithms can be used, such as described above. The training process continues until the model achieves a high accuracy in classifying optimal or sub-optimal cardiac resynchronization therapy.

The training and utilization of machine learning models to determine indications of patient condition, cardiac capture, etc., and to identify parameters or combinations of parameters associated with the desired clinical outcome, such as optimal or sub-optimal cardiac resynchronization therapy, etc., can depend on the specific type of ambulatory medical device being used and the nature of the physiological signal data it collects. Once trained, the machine learning model can receive new parameter settings and patient physiologic information as input and automatically detect if the patterns reflect the desired clinical outcome, such as optimal or sub-optimal cardiac resynchronization therapy, etc., and to identify specific parameter settings or combinations of parameter settings to change to achieve the desired clinical outcome. This allows advanced analysis without needing to hard-code detection criteria. The model can be periodically retrained on new data to optimize parameter settings over time.

In the inference stage of FIG. 5, for example, after the machine learning model 502 has been trained to reliably analyze parameter settings, patient physiologic information, or combinations thereof, and to identify and suggest programming or reprogramming recommendations to optimize a desired outcome or patient condition, such as cardiac resynchronization therapy, etc., the machine learning model 502 can be deployed for use in a production setting. Accordingly, existing parameter settings 503 (P1A, P1B, P2, . . . PN) of an ambulatory medical device (4) can be provided as input to the machine learning model 502, such as at a remote server computer (e.g., a cloud-based server or processor, etc.), in certain examples including patient physiologic information (O4). The machine learning model 502 can generate one or more outputs, including recommended parameter settings 504, indicating reprogramming recommendations for the ambulatory medical device (4A) and in certain examples an indication of outcome expected by such reprogramming recommendations (O4A).

In certain examples, the received patient physiologic information can be used as input to a rule-based recommendation engine for generating a recommendation to program or reprogram an ambulatory medical device, for example, when one or more parameter settings or combinations of parameter settings are identified by the machine learning analysis as suboptimal or having a high likelihood of resulting in suboptimal cardiac resynchronization therapy. In certain examples, a number of different parameter settings can be simulated to predict respective outcomes and the different parameter settings or combinations of parameter settings can be prioritized by the predicted respective outcomes.

In other examples, a desired outcome can be used as input to the rule-based recommendation engine for generating a recommendation to program or reprogram the ambulatory medical device. Different parameter settings can be simulated, in certain examples on different ambulatory medical devices, to predict respective outcomes. The predicted respective outcomes can be compared to the input desired outcome, and different parameter settings or combinations of parameter settings or different types of ambulatory medical devices, configurations, or therapies can be recommended based on a comparison of the predicted respective outcomes to the input desired outcome.

### Programming Recommendations

Consistent with some embodiments, a programming or reprogramming recommendation may involve a recommendation to modify or reprogram the device to use one or more different settings in sensing events, activity, or physiologic information, such as one or more blanking periods, thresholds, etc., or in providing stimulation, such as one or more intervals, delays, stimulation amplitudes, selected electrodes or vectors, etc. In certain examples, the recommendation to modify or reprogram the device can include a recommendation to use one or more different sensitivity settings or modes different from the current sensitivity setting or mode. With some embodiments, each sensitivity setting, or sensitivity mode is associated with one or more predefined threshold values. Accordingly, when a device is programmed or reprogrammed to operate in a new sensitivity mode, one or more of the predefined threshold values can change, thereby increasing or decreasing the sensitivity for detecting a particular event or activity. The programming or reprogramming recommendation may be presented via a user interface of a software application to a clinician, who may undertake the task of reprogramming the device for a patient. The clinician can then evaluate the recommendation and program or reprogram the ambulatory medical device accordingly. This allows the clinician to validate any proposed changes to the device based on their expert judgment. In other examples, the programming or reprogramming recommendation can be automatically applied within limits, such as previously validated by the clinician, etc., or directed based on physiologic response of the patient, such as determined by one or more patient response metrics indicative of patient response to different cardiac rhythm management therapy.

Embodiments described herein include numerous technical advantages for optimizing programming of and therapy delivery by ambulatory medical devices. By periodically evaluating collected physiological signal data using advanced machine learning models, closed-loop optimization of operation of the ambulatory medical device can be enabled over time. This allows enhancing operation of the ambulatory medical device compared to relying solely on static detection settings programmed at implantation. The machine learning analysis can identify suboptimal parameter settings missed by the ambulatory medical device or the clinician programming the ambulatory medical device and recommend adjustments to improve operation when appropriate. The system can adapt parameter settings to the individual patient commensurate with patient status or therapy efficacy, providing more accurate therapy and without requiring constant manual reprogramming, reducing workload for clinicians while optimizing device performance and increasing the speed of training and sharing updated protocols and guidance.

### Parameter Settings

Parameter settings differ based on the type of medical devices and in certain examples can include different modes of therapy. For example, different modes of cardiac resynchronization pacing include DDD and DDDR pacing, among others. The first “D” in DDD pacing represents dual (D) chamber (atrium and ventricle) pacing, the second “D” represents dual (D) chamber sensing, and the third “D” represents dual (D) chamber response to sensing in coordinating contraction of the heart and improve cardiac function. The additional “R” in DDDR pacing represents rate (R) modulation, where the medical device can adjust the pacing rate based on physiologic information indicating activity or need (e.g., activity, breathing rate, etc.).

Parameter settings can include, among others: Sensed Atrioventricular Delay Offset (SenAVDIyOffset or SAVDO), which adjusts the delay after a sensed atrial event; Atrioventricular Dynamic Minimum (AVDynMin or AVDM), which sets the minimum dynamic atrioventricular delay; Atrioventricular Delay Fixed (AVDlyFix or AVDF), which is a fixed atrioventricular delay setting; Atrioventricular Dynamic Maximum (AVDynMax or AVDM), which defines the maximum dynamic atrioventricular delay; Lower Rate Interval (LRLIntvl or LRLI), which sets the minimum pacing rate for the device; Left Ventricular Offset (LVOffset or LVO), which adjusts the timing of left ventricular pacing in relation to right ventricular pacing; Atrioventricular Dynamic Enable (AVDynEnbl or AVDE), which enables dynamic adjustment of the AVD; and Maximum Tracking Rate Interval (MTRIntvl or MTRI), the fastest interval at which the device will track atrial rates.

Additional parameter settings can include: Atrial Tachy Response Mode (ATRMode or ATRM), which defines the operational mode of the atrial channel; Biventricular Trigger Enable (BiVTrigEnbl or BVTE), which activates the biventricular pacing trigger; Ventricular Tachycardia Zone Rate (VTZoneRate or VTZR), which sets the rate threshold for detecting ventricular tachycardia; Atrial Tachy Response Trigger Rate (ATRTrigRt or ATRTR), which is the rate at which Atrial Tachy Response Mode is triggered; Maximum Sensor Rate Interval (MSRIntvl or MSRI), the maximum rate at which the device will pace in response to sensor input; Ventricular Tachycardia 1 Zone Rate (VT1ZoneRate or VT1ZR), which specifies the rate for a particular zone of ventricular tachycardia detection; Number of Ventricular Zones (NumVZones or NVZ), which determines how many zones are used for ventricular tachyarrhythmia detection; Ventricular Fibrillation Zone Rate (VFZoneRate or VFZR), which sets the rate threshold for detecting ventricular fibrillation; Atrial Tachy Response Ventricular Rate Regulation Response (ATRVRRResp or ATRVRRR), a setting that adjusts the ventricular pacing rate in response to atrial rate; Atrial Tachy Response Biventricular Trigger Enable (ATRBiVTrigEnbl or ATRBVTE), which allows for biventricular pacing in response to atrial rate; Atrial Tachy Response Lower Rate Limit (ATRLRL), which sets the minimum pacing rate in ATR mode; Tachycardia Mode (TachyMode or TM), which defines the operational mode for tachycardia detection and therapy; Respiration Rate Trend Enable (RRTenable or RRT), which activates the respiration rate tracking feature; Atrial Tachy Response Pacing Chamber (ATRPaceCham or ATRPC), which specifies the chamber to be paced in atrial tachy mode; and Sensing Mode (SenseMode), which determines how the device senses cardiac events.

### Related Parameter Settings

The different parameter settings described above can have different having different relationships with respect to each other. For example, based on analysis of multiple patients using feature extraction, such as illustrated and described in FIG. 4, and machine learning, such as described in FIG. 5, different combinations or subcombinations can be determined having closer relationships than others, such that if one parameter is going to be adjusted, the patient is likely to benefit from adjustment of a second or one or more other parameters. Different subgroups are illustrated below, the first having a closer relationship than the second, the second closer than the third, etc.

A first subgroup can include the following parameters, listed in order of relationship (e.g., parameters (1) and (2) are closer than (1) and (9), etc.), in certain examples enforced using one or more rules executed by the programmer: (1) Sensing Mode (SenseMode) and Atrial Tachy Response Pacing Chamber (ATRPaceCham or ATRPC) should be adjusted or recommended to be adjusted together, for example, on the same or sequential programming screens of the programmer, followed closely by; (2) Respiration Rate Trend Enable (RRTenable or RRT); (3) Tachycardia Mode (TachyMode or TM), (4) Atrial Tachy Response Lower Rate Limit (ATRLRL). In certain examples, the first subgroup can additionally include the following parameters: (5) Atrial Tachy Response Ventricular Rate Regulation Response (ATRVRRResp or ATRVRRR) and (6) Atrial Tachy Response Biventricular Trigger Enable (ATRBiVTrigEnbl or ATRBVTE). Somewhat unexpectedly, (7) Number of Ventricular Zones (NumVZones or NVZ); (8) Ventricular Fibrillation Zone Rate (VFZoneRate or VFZR); and (9) Ventricular Tachycardia 1 Zone Rate (VT1ZoneRate or VT1ZR) are more closely related to each other than (1)-(6).

A second subgroup can include the first subgroup (e.g., parameters (1)-(9)) and additionally the following: (10) Maximum Sensor Rate Interval (MSRIntvl or MSRI); (11) Atrial Tachy Response Trigger Rate (ATRTrigRt or ATRTR); (12) Ventricular Tachycardia Zone Rate (VTZoneRate or VTZR); (13) Biventricular Trigger Enable (BiVTrigEnbl or BVTE); and (14) Atrial Tachy Response Mode (ATRMode or ATRM).

A third subgroup can include the following parameters: Atrioventricular Delay Fixed (AVDlyFix or AVDF) and Atrioventricular Dynamic Maximum (AVDynMax or AVDM), suggesting that such parameter settings should be grouped using one or more rules. A fourth subgroup can include the third subgroup and additionally the following parameters: Sensed Atrioventricular Delay Offset (SenAVDIyOffset or SAVDO) and Atrioventricular Dynamic Minimum (AVDynMin or AVDM). Each of the identified subgroups have significant identified relationships and can be changed or alerted to be changed together, such that if one setting from the group is adjusted, the others are alerted for consideration or adjustment.

As described herein, patterns or combinations of parameter settings can be identified and validated as affecting or impacting cardiac resynchronization therapy. For example, groups of parameters can be identified having a higher percentage of cardiac capture rates than other parameters, or groups that, once such parameter settings are implemented, show an increase in rates of cardiac capture. Once identified, individual values for specific parameter settings can be toggled or changed and the impact to cardiac resynchronization therapy across a population or number of patients can be analyzed to determine positive or negative impact on delivered therapy, such as evidenced by rates of cardiac capture or other patient physiologic information.

In one example, a first group of parameter settings was identified as: VTZoneRate=300 ms, SenAVDlyOffset=40 ms, NumVZones=3. Once implemented, an increase in LV cardiac capture was detected. To validate the identified group, cardiac capture rates (e.g., LV pacing % (successful capture) and RV pacing %) are analyzed by toggling values of the parameter settings to or away from default or mode values for each setting and comparing cardiac capture rates for different values. If the cardiac capture rates do not significantly change (e.g., increase or decrease less than a threshold percentage) with respect to the toggle, the specific parameter value can be identified as not having a substantial impact. However, if the cardiac capture rates change (e.g., an increase or decrease more than a threshold percentage) with respect to the toggle, the specific parameter value can be identified as having a substantial impact. In this example, the VTZoneRate=300 ms parameter (e.g., toggled with respect to an example mode value of 400 ms, etc.) provided a more substantial positive impact on LV cardiac capture rate than the other parameters, though less than the combination of the first group of parameter settings in aggregate, validating the combination.

In contrast, in another example, a second group of parameter settings was identified as: BiVTrigEnbl=1 , SenAVDlyOffset=50 ms, VFZoneRate=273 ms, NumVZones=3. Once implemented, an increase in RV cardiac capture was detected. To validate the identified group, cardiac capture rates are analyzed by toggling values of the parameter settings to or away from default or mode values for each setting and comparing cardiac capture rates for different values. In this example, the BiVTrigEnbl=1 parameter (e.g., toggled from 1 to 0) provided a substantial positive impact on LV cardiac capture and the NumVZones=3 parameter (e.g., toggled from 3 to 2, etc.) provided a substantial positive impact on RV cardiac capture, though each less than the combination of the second group of parameter settings in aggregate, validating the combination.

### CRT vs. MSP Mode Switch

Certain heart failure patients respond to (e.g., benefit from) multi-site pacing therapy that do not respond to traditional (non-multi-site pacing) cardiac resynchronization therapy. Other patients do not respond to either. Pacing therapies can be evaluated to ensure that the applied pacing therapy is providing some benefit to the patient, to determine whether or not the pacing therapy should be adjusted, or to determine if the pacing therapy should be transitioned. For example, one study found that 25% to 40% of heart failure patients with myocardial dysfunction did not respond to conventional cardiac resynchronization therapy (e.g., biventricular pacing therapy) by evaluation at 6 months post implant, but also found that over half (51.3%) of non-responders to cardiac resynchronization therapy at 6 months did subsequently respond to 6 months of multi-site pacing therapy in the left ventricle by evaluation at 12 months post implant.

Although the transition from CRT to multi-site pacing therapy in the left ventricle was substantially complication free physiologically, the switch to multi-site pacing therapy from conventional CRT can provide at least some cardiac stress, but also requires additional resources from the implantable medical device. In certain estimates, implementing multi-site pacing therapy in a device capable of multi-site pacing therapy and CRT reduces the estimated life of the device by 11-13%. Even a single six-month evaluation of multi-site pacing therapy on an IMD can impact the usable life of the IMD by a relatively substantial and often unnecessary amount.

In certain examples, physiologic information sensed from the patient in one or more time periods can be used to determine one or more multi-site pacing response metrics, and that such one or more determined multi-site pacing response metrics can be used to provide an alert or notification or otherwise control transition between different medical device modes (e.g., a first stimulation mode, a second stimulation mode, etc.), including, in certain examples, to: (1) control transition of a non-multi-site pacing therapy mode to a multi-site pacing therapy mode; (2) control transition of the multi-site pacing therapy mode to the non-multi-site pacing therapy mode; or (3) enable or disable the multi-site pacing therapy mode.

Moreover, physiologic information sensed from the patient subsequent to implementing a stimulation mode can be used to determine a multi-site pacing response metric configured to determine an indication of a predicted patient response to the stimulation mode, such that, in certain examples, a stimulation mode can be evaluated without entering the stimulation mode. For example, specific physiologic information or combinations of physiologic information can be determined to evaluate a stimulation mode or to predict a positive patient response to a particular stimulation mode, such as using a response to another stimulation mode, etc. In an example, physiologic information sensed or detected during a CRT mode can be used to determine if a patient is likely to respond to (e.g., benefit from) a multi-site pacing therapy mode before the multi-site pacing therapy mode is implemented or enabled.

In an example, impedance information (e.g., ITTI), respiration information (e.g., RSBI), or a combination thereof over one or more time periods can be used to determine an indication that a patient will respond to a multi-site pacing therapy mode. A first patient response metric, p1, (e.g., a multi-site pacing response metric) can be determined as follows:

(α×ITTI)+(β×RSBI) (1)

In equation (1), ITTI is a measure of patient intrathoracic impedance, RSBI is a measure of patient respiratory rate (RR) or frequency to a measure of tidal volume (TV) (e.g., RR/TV, etc.), and α and β are variables. In other examples, the patient response metric can be determined using only one of ITTI or RSBI information, or as different functions of one or more of such physiologic parameters in combination with one or more other physiologic parameters.

In other examples, other patient response metrics can be determined using other physiologic information, such as heart sound information (e.g., S3/S1, etc.), RSBI information, ITTI information, etc. For example, S3/S1, is particularly well correlated to determining if a patient is likely to respond to a CRT mode (or CRT mode or MSP therapy mode), in contrast to being a non-responder (e.g., there is a difference in heart sound information between responders and non-responders). RSBI information is similarly well correlated. A second patient response metric, p2, (e.g., a CRT pacing response metric) can be determined as follows:

(α×(S3/S1))+(β×RSBI) (2)

In equation (2), S3 is a third heart sound parameter (e.g., an amplitude or energy of the third heart sound, etc.), S1 is a first heart sound parameter (e.g., an amplitude or energy of the first heart sound, etc.), RSBI is a measure of patient respiratory rate (RR) or frequency to a measure of tidal volume (e.g., RR/TV, etc.), and α and β are variables. In other examples, the patient response metric can be determined using a combination of this or other heart sound information (e.g., other than S3/S1, etc.), using only one of heart sound or RSBI information, using information over different time periods, or as a combination with one or more other physiologic parameters.

In other examples, one or more other patient response metrics can be determined using sensed or received physiologic information of the patient, or combinations of sensed or received physiologic information of the patient, such as one or more of night heart rate information, activity information, determination of patient rest (e.g., a lack of activity, etc.), a multi-sensor HeartLogic™ index, a slope of a minute ventilation (MV) signal, determined AVD parameters, pacing thresholds in or in response to the different therapies or modes, continuous electrogram measurements, impedance measurements, occurrence or detection of adverse events (e.g., arrhythmia, etc.), etc. Different modes or parameter settings can be implemented for a time period (e.g., 1 week, 1 month, etc.), the patient response metric can be determined in response thereto, and the therapy can be adjusted accordingly, with reporting and alerts coincident to changes in determined patient response metrics, with changes in parameter settings, or in response to changes exceeding one or more thresholds. In certain examples, patient response metrics can include a comparison of a determined patient response metric pre and post change and can trigger further adjustment or a report to a clinician for review.

### Medical Device Systems

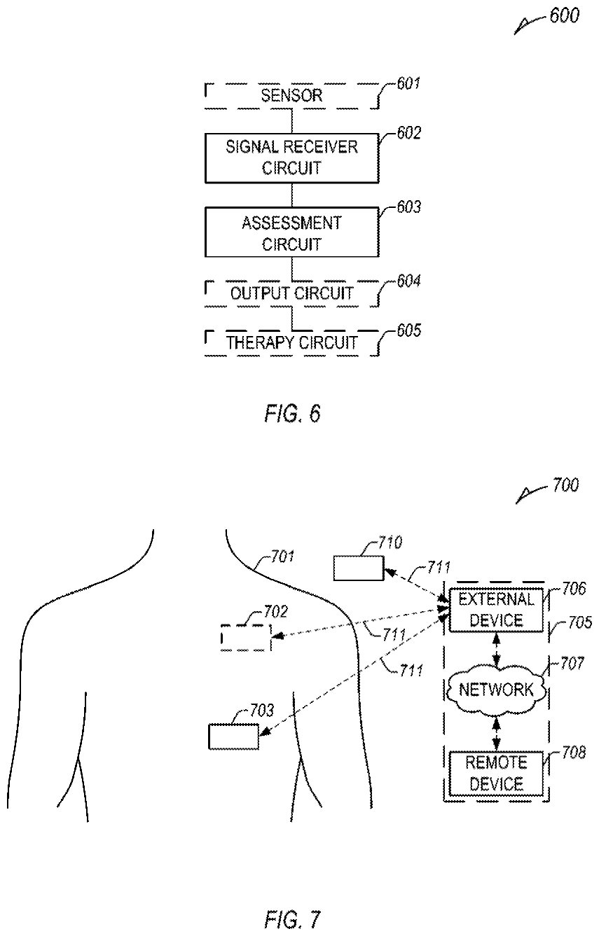

FIG. 6 illustrates an example system 600 (e.g., a medical device system). In an example, one or more aspects of the system 600 can be a component of, or communicatively coupled to, a medical device, such as an implantable medical device (IMD), an insertable cardiac monitor, an ambulatory medical device (AMD), etc. The system 600 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 600 can include a single medical device or a plurality of medical devices implanted in a body of a patient or otherwise positioned on or about the patient to monitor patient physiologic information of the patient using information from one or more sensors, such as a sensor 601. In an example, the sensor 601 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, a potassium sensor, a creatinine sensor, etc.); a temperature sensor; a skin elasticity sensor, or one or more other sensors configured to receive physiologic information of the patient.

The example system 600 can include a signal receiver circuit 602 and an assessment circuit 603. The signal receiver circuit 602 can be configured to receive physiologic information of a patient (or group of patients) from the sensor 601. The assessment circuit 603 can be configured to receive information from the signal receiver circuit 602, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. Physiologic information can include, among other things, one or more of: electrical information of the patient, such as cardiac electrical information (e.g., heart rate, heart rate variability, etc.), impedance information, temperature information, and in certain examples, respiration information (e.g., a respiratory rate, a respiration volume (tidal volume), etc.); mechanical information of the patient, such as cardiac acceleration information (e.g., cardiac vibration information, pressure waveform information, heart sound information, endocardial acceleration information, acceleration information, activity information, posture information, etc.), physical activity information (e.g., activity, steps, etc.), posture or position information, pressure information, plethysmograph information, and in certain examples, respiration information; chemical information; or other physiologic information of the patient. In an example, the signal receiver circuit 602 can include the sensor 601. In other examples, the signal receiver circuit can be coupled to or a component of the assessment circuit 603.

In certain examples, the assessment circuit 603 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 600 can include an output circuit 604 configured to provide an output to a user, or to cause an output to be provided to a user, such as through an output, a display, or one or more other user interface, the output including a score, a trend, an alert, or other indication. In other examples, the output circuit 604 can be configured to provide an output to another circuit, machine, or process, such as a therapy circuit 605 (e.g., a cardiac resynchronization therapy circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more cardiac resynchronization therapy parameters, drug delivery, dosage determinations or recommendations, etc. In an example, the therapy circuit 605 can include one or more of a stimulation control circuit, a cardiac stimulation circuit, a neural stimulation circuit, a dosage determination or control circuit, etc. In other examples, the therapy circuit 605 can be controlled by the assessment circuit 603, or one or more other circuits, etc. In certain examples, the assessment circuit 603 can include the output circuit 604 or can be configured to determine the output to be provided by the output circuit 604, while the output circuit 604 can provide the signals that cause the user interface to provide the output to the user based on the output determined by the assessment circuit 603. In other examples, the assessment circuit 603 can include the therapy circuit 605, a memory circuit, or one or more other circuits or components.

In certain examples, one or more of the sensor 601, the signal receiver circuit 602, the assessment circuit 603, the output circuit 604, or the therapy circuit 605 can include a communication circuit or be configured to control a communication circuit of one or more other circuit, device, or component.

### Efficiency and Mode Transitions

Ambulatory medical devices powered by rechargeable or non-rechargeable batteries, responsible for sensing physiologic signals and physiologic information of the patient, and in certain examples making determinations using such information, have to make certain tradeoffs between device battery life, or in the instance of implantable medical devices with non-rechargeable batteries, between device replacement periods often including surgical procedures, and device sensing, storage, processing, and communication characteristics, such as sensing resolution, sampling frequency, sampling periods, the number of active sensors, the amount of stored information, processing characteristics, or communication of physiologic information outside of the device.

Medical devices can include higher-power modes and lower-power modes. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode.

A technological problem in the art with respect to such devices exists that not all information can be stored, not all sensors can be active in a high-power or high-resolution mode, not all algorithms can be active, and not all sensed or processed information can be communicated outside of the device at all times without detrimentally impacting the lifespan of the devices. Technological solutions to such problems are often improvements in physical sensors, or alternatively in sensing and processing physiologic information in a way that improves device efficiency, extending the lifespan of the device, or to perform new determinations using existing sensors or information in a way that was not previously known, increasing the capabilities of an existing device without adding additional hardware to the device, or requiring additional sensors or hardware to be implanted in the patient. Efficiency improvements in one area can enable additional operation in another, improving the technical capabilities of existing devices having real-world constraints.

For example, physiologic information, such as indicative of a potential adverse physiologic event, can be used to transition from a low-power mode to a high-power mode. However, by the time physiologic information detected in the low-power mode indicates a possible event, valuable information has been lost, unable to be recorded in the high-power mode.

Another technological problem exists in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

In an example, a change in modes can enable higher resolution sampling or an increase in the sampling frequency or number or types of sensors used to sense physiologic information leading up to and including a potential event. Different physiologic information is often sensed using non-overlapping time periods of the same sensor, in certain examples, at different sampling frequencies and power costs.

For example, ambulatory medical devices frequently contain one or more accelerometer sensors and corresponding processing circuits to determine and monitor patient acceleration information, such as, among other things, cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiratory rate (RR), tidal volume (TV), rapid shallow breathing index (RSBI), respiration phase, breathing sounds, etc.), etc. In one example, heart sounds and patient activity can be detected using non-overlapping time periods of the same, single-or multi-axis accelerometer, at different sampling frequencies and power costs.

In an example, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

Another technological problem exists in that suboptimal programming of device parameters and parameter settings can negatively impact functionality of ambulatory medical devices. Accordingly, identifying suboptimal programming by clinicians and other caregivers and generating and providing alerts or notifications of such identified suboptimal programming, or reprogramming recommendations, and in certain examples, reprogramming ambulatory medical devices directly, can improve the functionality of existing ambulatory medical devices without requiring other improvements to the hardware of devices providing therapy or the sensors themselves.

### Physiologic Parameters

Heart sounds are recurring mechanical signals associated with cardiac vibrations or accelerations from blood flow through the heart or other cardiac movements with each cardiac cycle and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

Respiration information can include, among other things, a respiratory rate (RR) of the patient, a tidal volume (TV) of the patient, a rapid shallow breathing index (RSBI) of the patient, or other respiratory information of the patient. The respiratory rate is a measure of a breathing rate of the patient, generally measured in breaths per minute. The tidal volume is an aggregate measure of respiration changes, such as detected using measured changes in thoracic impedance, etc. The RSBI is a measure (e.g., a ratio) of respiratory frequency relative to (e.g., divided by) tidal volume of the patient. The nHR is a measure of heart rate (HR) of the patient at night, either in relation to sensing patient sleep or using a preset or selectable time of day corresponding to patient sleep. In certain examples, respiration information of the patient can be determined using changes in impedance information and accordingly can be considered electrical information, but different than cardiac electrical information. In other examples, respiration information of the patient can be determined using changes in activity or acceleration information and accordingly can be considered mechanical information.

Physiologic metrics, as described herein, or measures or indications of physiologic information, can include one or more different measures of rate, amplitude, energy, etc., of different physiologic information over one or more time periods, such as representative daily values, etc. For example, heart sound metrics can be determined for each heart sound (e.g., the first heart sound (S1) through the fourth heart sound (S4), etc.) and can include an indication of an amplitude or energy of a specific heart sound for a specific cardiac cycle, or a representation of a number of cardiac cycles of the patient over a specific time period. Daily metrics can be determined representative of an average daily value for the patient, either corresponding to a waking time or a 24-hour period, etc. Respiration metrics can include, among other things, a mean or median respiratory rate, binned values of rates, and a representative value of specific rate bins, etc. Heart rate metrics can include an average nighttime heart rate, a minimum nighttime heart rate, heart rate at rest, etc.

The activity information can include an activity measurement of the patient, such as detected using an accelerometer, a posture sensor, a step counter, or one or more other activity sensors associated with an ambulatory medical device. Activity may be used to gate other physiologic measurements such as heart rate or respiratory rate so that the change in these metrics with increased patient activity may be used to infer patient cardiovascular and metabolic status including measurement of oxygen consumption. The impedance information can include, among other things, thoracic impedance information of the patient, such as a measure of impedance across a thorax of the patient from one or more electrodes associated with the ambulatory medical device (e.g., one or more leads of an implantable medical device proximate a heart of the patient and a housing of the implantable medical device implanted subcutaneously at a thoracic location of the patient, one or more external leads on a body of the patient, etc.). In other examples, the impedance information can include one or more other impedance measurements associated with the thorax of the patient, or otherwise indicative of patient thoracic impedance.

The temperature information can include an internal patient temperature at an ambulatory medical device, such as implanted in the thorax of the patient, or one or more other temperature measurements made at a specific location on the patient, etc. The temperature information can be detected using a temperature sensor, such as one or more circuits or electronic components having an electrical characteristic that changes with temperature. The temperature sensor can include a sensing element located on, at, or within the ambulatory medical device configured to determine a temperature indicative of patient temperature at the location of the ambulatory medical device.

In contrast to and separate from the electrical or mechanical information discussed above, the chemical information can include information about one or more chemical properties of blood, interstitial space (e.g., the space between cells, such as including interstitial fluid), or other tissue (e.g., muscle tissue, fat tissue, organ tissue, etc.) of the patient, such as information indicative of or including one or more of a glucose level, pH level, dissolved gas level (e.g. oxygen, carbon dioxide, carbon monoxide, etc.), electrolyte level (e.g., sodium, potassium, calcium, etc.), organic compound level (e.g., lactate, cholesterol, hemoglobin, creatinine, etc.), or biologic compound level (e.g., enzymes, antibodies, receptors, etc.), etc. The chemical information may be measured by one or more of an electrical sensor, mechanical sensor, electrochemical sensor, biosensor (e.g., enzyme biosensor, etc.), ion-selective electrode sensor, optical sensor, etc. In an example, the chemical information may include potassium information (e.g., one or more of interstitial potassium information, serum potassium information, etc.), creatinine information (e.g., one or more of interstitial creatinine information, serum creatinine information, etc.), or combinations thereof.

In certain examples, interstitial chemical information, such as one or more chemical levels in an interstitial space (e.g., a space between one or more of connective tissue, muscle fibers, nervous tissue, etc.) or of interstitial fluid, etc., can be indicative of serum chemical information. For example, potassium may move between cells or tissue and interstitial fluid (e.g., a change in interstitial potassium level may be followed by or reflective of a change in serum potassium level or vice versa), such that chemical information on serum potassium can include interstitial potassium. In certain examples, one of interstitial or serum chemical information can lead or lag the other, such that a change in one can indicate a worsening patient condition is detectable before the other. In one example, interstitial potassium information can lead serum potassium information as an indicator of electrolyte imbalance.

### Alert States

In certain examples, an alert state (e.g., an in-alert state, an out-of-alert state, a priority alert state, etc.) of the patient can be adjusted or determined using chemical information of the patient, such as to increase a sensitivity or specificity of alert state determination, reduce false positive alert state determinations, alert state transitions or adjustments, or otherwise reduce storage or transmission of physiologic information associated or transitions associated with false positive alert state determinations, and power and processing resources associated with the same. In an example, the alert state can be determined using a comparison of a value of the health index (e.g., a numerical value, etc.) to one or more fixed or adaptable alert thresholds (e.g., based at least in part on one or more relative factors, such as measurements from the patient over the past 30 days, etc.). In an example, the alert state can be provided to a user interface for display to a user or to a control circuit to control or adjust a process or function of the system. In an example, the alert state can include one or more of an indication, recommendation, or instruction to perform one or more actions (e.g., administer or provide a drug or class of drug, adjust or optimize a guideline-directed medical therapy (GDMT), etc.). For, example, a GDMT may advise administration of a quantity of a drug or a rate of increase in a dosage, etc. In an example, determination of an in-alert or priority alert state can trigger an indication or instruction to administer or provide a specific class of diuretic or to deviate from GDMT (e.g., increase GDMT above a standard recommendation, hold GDMT at a standard recommendation, hold GDMT at a current level, decrease GDMT below a standard recommendation, increase a dosage or rate of increase of a drug, reduce a dosage or rate of decrease of a drug, etc.).

In certain examples, the techniques described above or herein can be used in various combinations or permutations. For example, combinations or permutations of techniques described above or herein can be selected based upon patient history, patient treatment (e.g., in-patient care, out-patient care, etc.), clinician input, etc.

As used herein, high and low (or high, medium, and low, etc.) can be relative or categorical terms, in certain examples with respect to clinical or population values, patient-specific values (e.g., a representative value, such as a current value, with respect to a short- or long-term range of values, etc.), or combinations thereof. For example, a high value can include a value in an upper percentage (e.g., at or above an upper quartile, etc.) of values experienced by the patient over respective time periods, such as one or more of a short-term range (e.g., having a period between 1 week and 3 months, such as 1 month, etc.), a long term range (e.g., having a period greater than the short-term range, such as greater than 1 month, greater than 3 months, the last 6 months, or longer, etc.). A low value can include a value in a lower percentage (e.g., at or below a mean or median, below the upper quartile, etc.). A medium value can, in certain examples, include a value between the upper and lower quartiles or within a threshold percentage of a mean or median, etc. In other examples, values can be determined with respect to clinical or population values, in certain examples, further respective to matching patient demographics (e.g., age, sex, comorbidities, etc.) or type of medical device (e.g., CRT-D device, ICD device, etc.), etc.

In an example, determinations described herein can be used to change device behavior, trigger additional sensing, data processing, storage, or transmission, or otherwise alter one or more modes, processes, or functions of medical devices associated with such determinations. For example, determinations can require data over a substantial time period (e.g., multiple days, weeks, a month or more, etc.). Such determinations can be initially determined by the device at yearly or semi-yearly (e.g., every 6 months, every 3 months, etc.) by default, or triggered by worsening patient status or upon instruction from a clinician or caregiver, etc. In a first example, an assessment circuit can determine one or more indications quarterly, consuming a default amount of device resources. If the quarterly determination exceeds one or more of a patient-specific or population threshold, the assessment circuit can alter device functionality to increase the frequency of making such determinations, increasing the use of device resources, in certain examples reducing device lifespan, but providing additional monitoring and determinations. In other examples, if a determination exceeds one or more thresholds, additional sensing can be triggered, such as enabling additional sensors, or sensing enabled sensors with a higher resolution or sampling frequency, storing more information, and communicating more information outside of the device, such as to an external programmer, or increasing the frequency of communication outside of the device, increasing the use of device resources, in certain examples reducing device lifespan, but providing additional monitoring and determinations.

In certain examples, determinations described herein can include one or more determined risk curves illustrating determined risks at different time periods into the future, such as a determined risk of mortality (e.g., cardiovascular death), a determined risk of heart failure hospitalization, etc. Information about the determined risks or the determined risk curves or portions of the determined risk curves themselves can be provided to a user, such as to a patient, clinician, caregiver, etc., or can be used to make one or more device changes, such as described herein (e.g., therapies, treatments, device settings, etc.), or trigger one or more other processes or notifications, etc.

### Patient Indications

Indications of patient condition (e.g., improved or worsening patient condition, etc.) can include single-feature determinations based on a single feature or measure of a single type of physiologic information, or separately a composite determination based on a combination of physiologic information, such as two or more separate features of physiologic measures. In addition, indications of patient condition can be device-based, such as determined using physiologic information detected from the patient using the one or more ambulatory medical devices without input of clinical information about the patient separate from that detected or sensed physiologic information. In other examples, indications of patient condition can be a combination of device-based and clinical-based information of the patient, such as clinician diagnosis or determination of risk, patient history, patient age, comorbidities, prior hospitalization, type of implanted device, etc. In certain examples, separate determinations can be made for different combinations of clinical information.

One example of a composite indication is the HeartLogic™ index, a HeartLogic™ in-alert time, or one or more other composite measurements or measures thereof. The HeartLogic™ index is a composite indication of patient condition determined using different combinations or weightings of physiologic information, including two or more of S1 heart sounds, S3 heart sounds, thoracic impedance, activity information, respiration information, and nighttime heart rate (nHR). The HeartLogic™ index can be indicative of a heart failure status, a risk a heart failure event (e.g., within in a given time period), or a worsening of the heart failure status or risk of heart failure event in the patient over time. The HeartLogic™ in-alert time is a measure of time that the HeartLogic™ index is above an alert threshold.

In certain examples, the different combinations or weightings of physiologic information used to determine the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.). For example, if the risk stratifier is low, or below a first threshold, the HeartLogic™ index can be determined using a first combination of physiologic information. If the risk stratifier is high, or above a second threshold, the HeartLogic™ index can be determined using a second combination of physiologic information, such as additional information than included in the first combination (e.g., the first combination and the second combination, etc.). If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination than the first combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

### Patient Management System

FIG. 7 illustrates an example patient management system 700 and portions of an environment in which the patient management system 700 may operate. The patient management system 700 can perform a range of activities, including remote patient monitoring and diagnosis of a disease condition, programming of ambulatory medical devices, and control of one or more therapies. Such activities can be performed proximal to a patient 701, such as in a patient home or office, through a centralized server, such as in a hospital, clinic, or physician office, or through a remote workstation, such as a secure wireless mobile computing device.

The patient management system 700 can include one or more medical devices, an external system 705, and a communication link 711 providing for communication between the one or more ambulatory medical devices and the external system 705. The one or more medical devices can include an ambulatory medical device, such as an implantable medical device 702, a wearable medical device 703, or one or more other implantable, leadless, subcutaneous, external, wearable, or medical devices configured to monitor, sense, or detect information from, determine physiologic information about, or provide one or more therapies to treat various conditions of the patient 701, such as one or more cardiac or non-cardiac conditions (e.g., dehydration, sleep disordered breathing, etc.).

In an example, the implantable medical device 702 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 701. In another example, the implantable medical device 702 can include a monitor implanted, for example, subcutaneously in the chest of patient 701, the implantable medical device 702 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured to detect physiologic information from the patient, or provide one or more therapies or stimulation to the patient.

Implantable devices can additionally or separately include leadless cardiac pacemakers, small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemaker devices can be implanted in or about the heart to detect physiologic information from, or provide one or more therapies or stimulation to, one or more chambers of the heart. The multiple leadless cardiac pacemaker devices can communicate between themselves, or one or more other implanted or external devices.

The implantable medical device 702 can include a signal receiver circuit or an assessment circuit configured to detect or determine specific physiologic information of the patient 701, or to determine one or more conditions or provide information or an alert to a user, such as the patient 701 (e.g., a patient), a clinician, or one or more other caregivers or processes, such as described herein. The implantable medical device 702 can alternatively or additionally be configured as a therapeutic device configured to treat one or more medical conditions of the patient 701. The therapy can be delivered to the patient 701 via the lead system and associated electrodes or using one or more other delivery mechanisms. The therapy can include delivery of one or more drugs to the patient 701, such as using the implantable medical device 702 or one or more of the other ambulatory medical devices, etc. In some examples, therapy can include cardiac resynchronization therapy for rectifying dyssynchrony and improving cardiac function in heart failure patients. In other examples, the implantable medical device 702 can include a drug delivery system, such as a drug infusion pump to deliver drugs to the patient for managing arrhythmias or complications from arrhythmias, hypertension, hypotension, or one or more other physiologic conditions. In other examples, the implantable medical device 702 can include one or more electrodes configured to stimulate the nervous system of the patient or to provide stimulation to the muscles of the patient airway, etc.

The wearable medical device 703 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 705 can include a dedicated hardware/software system, such as a programmer, a remote server-based patient management system, or alternatively a system defined predominantly by software running on a standard personal computer. The external system 705 can manage the patient 701 through the implantable medical device 702 or one or more other ambulatory medical devices connected to the external system 705 via a communication link 711. In other examples, the implantable medical device 702 can be connected to the wearable medical device 703, or the wearable medical device 703 can be connected to the external system 705, via the communication link 711. This can include, for example, programming or reprogramming the implantable medical device 702 with different parameter settings to perform one or more of acquiring physiologic data, performing at least one self-diagnostic test (such as for a device operational status), analyzing the physiologic data, or optionally delivering or adjusting a therapy for the patient 701. Additionally, the external system 705 can send information to, or receive information from, the implantable medical device 702 or the wearable medical device 703 via the communication link 711. Examples of the information can include real-time or stored physiologic data from the patient 701, diagnostic data, such as detection of patient hydration status, hospitalizations, responses to therapies delivered to the patient 701, or device operational status of the implantable medical device 702 or the wearable medical device 703 (e.g., battery status, lead impedance, etc.). The communication link 711 can be an inductive telemetry link, a capacitive telemetry link, or a radio frequency (RF) telemetry link, such as a wireless telemetry based on, for example, Bluetooth® or IEEE 802.11 wireless fidelity “Wi-Fi” interfacing standards. Other configurations and combinations of patient data source interfacing are possible.

The external system 705 can include an external device 706 in proximity of the one or more ambulatory medical devices, and a remote device 708 in a location relatively distant from the one or more ambulatory medical devices, in communication with the external device 706 via a communication network 707. Examples of the external device 706 can include a medical device programmer. The remote device 708 can be configured to evaluate collected device or patient information and provide alert notifications, among other possible functions. In an example, the remote device 708 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 708 can receive data from multiple patients. The data can be collected by the one or more ambulatory medical devices, among other data acquisition sensors or devices associated with the patient 701. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text, or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

In an example, similar to the alert notifications discussed above, the external system 705 or one or more components thereof (e.g., the external device 706, the remote device 708, an assessment circuit, etc.) can be configured to schedule one or more follow-up appointments or adjust a schedule of one or more follow-up appointments for the patient such as in response to one or more alert notifications or other determinations, per a request of a clinician, etc.

The remote device 708 may additionally include one or more locally configured clients or remote clients securely connected over the communication network 707 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 708, including the server and the interconnected clients, may also execute a follow-up scheme by sending follow-up requests to the one or more ambulatory medical devices, or by sending a message or other communication to the patient 701 (e.g., the patient), clinician or authorized third party as a compliance notification.

The communication network 707 can provide wired or wireless interconnectivity. In an example, the communication network 707 can be based on the Transmission Control Protocol/Internet Protocol (TCP/IP) network communication specification, although other types or combinations of networking implementations are possible. Similarly, other network topologies and arrangements are possible.

One or more of the external device 706 or the remote device 708 can output the detected medical events to a system user, such as the patient or a clinician, or to a process including, for example, an instance of a computer program executable in a microprocessor. In an example, the process can include an automated generation of a programming recommendation for an ambulatory medical device to optimize or improve patient condition or otherwise provide a desired clinical outcome. In an example, the external device 706 or the remote device 708 can include a respective display unit for displaying the physiologic or functional signals, or alerts, alarms, emergency calls, or other forms of warnings to signal the detection of one or more conditions. In some examples, the external system 705 can include a signal receiver circuit and an assessment circuit, such as an external data processor configured to analyze the physiologic or functional signals received by the one or more ambulatory medical devices, and to confirm or reject one or more determinations made by one or more ambulatory medical devices, such as the implantable medical device 702, the wearable medical device 703, etc., or make additional determinations, etc. Computationally intensive algorithms, such as machine-learning algorithms, can be implemented in the external data processor.

With some examples, when parameter settings of an ambulatory medical device are analyzed using one or more trained machine learning models, and one or more differences between the parameter settings of the ambulatory medical device and the stored model parameter settings are detected, a recommendation to reprogram the medical device may be generated and presented to a clinician via a user interface of the remote device 708, or via a user interface of a software application executing on a client device communicatively connected with the remote device 708. The recommendation to reprogram the medical device may be determined by identifying differences between the parameter settings of the ambulatory medical device and the stored model parameter settings via the one or more machine learning models that otherwise went undetected by a clinician or a medical device programmer.

Portions of the one or more ambulatory medical devices or the external system 705 can be implemented using hardware, software, firmware, or combinations thereof. Portions of the one or more ambulatory medical devices or the external system 705 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

A therapy device 710 can be configured to send information to or receive information from one or more of the ambulatory medical devices or the external system 705 using the communication link 711. In an example, the one or more ambulatory medical devices, the external device 706, or the remote device 708 can be configured to control one or more parameters of the therapy device 710. The external system 705 can allow for programming or reprogramming the one or more ambulatory medical devices and can receive information about one or more signals acquired by the one or more ambulatory medical devices, such as can be received via a communication link 711. The external system 705 can include a local external implantable medical device programmer. The external system 705 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected events can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), in certain examples, in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, for example, to increase arterial pressure, to maintain cardiac output, to disrupt or reduce the impact of the detected event, or combinations thereof.

In certain examples, physiologic information of a patient can be sensed using one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite first heart sound (S1) parameter representative of a plurality of S1 parameters, for example, over a certain time period (e.g., a number of cardiac cycles, a representative time period, etc.), or one or more other heart sounds (e.g., a second heart sound (S2), a third heart sound (S3), a fourth heart sound (S4), etc.), etc.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient. In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected event (e.g., a heart failure event, an arrhythmia event, etc.) can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

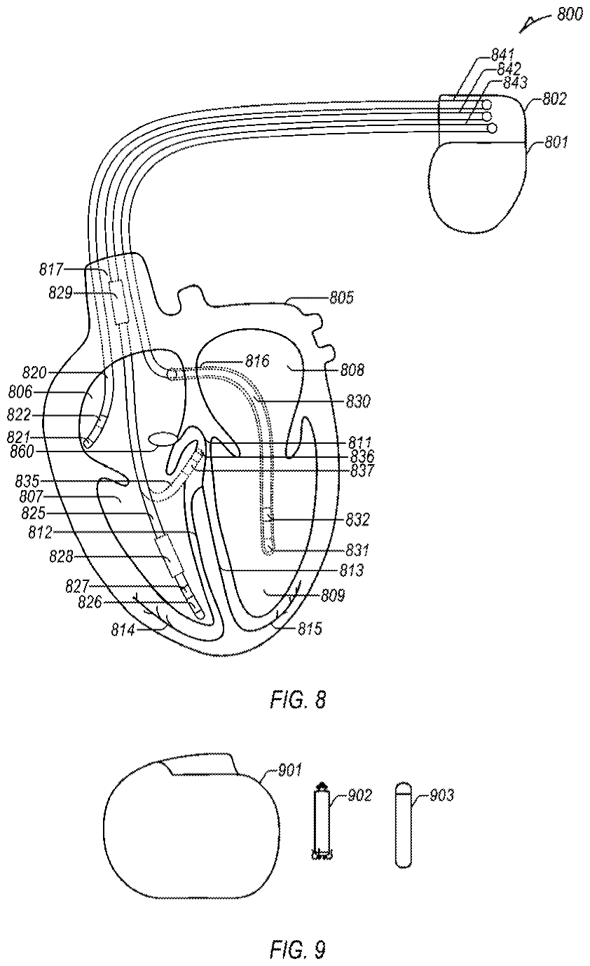

FIG. 8 illustrates an example implantable medical device system 800 including an implantable medical device 801 electrically coupled to a heart 805, such as through one or more leads coupled to the implantable medical device 801 through one or more lead ports in a header 802 of the implantable medical device 801. In this example, the one or more leads include first, second, and third leads 820, 825, 830 in traditional lead placements in the right atrium (RA) 806, right ventricle (RV) 807, and in a coronary vein 816 (e.g., the coronary sinus) over the left atrium (LA) 808 and left ventricle (LV) 809, respectively, and a fourth lead 835 positioned in the RV 807 near the His bundle 811, between the AV node 810 and the right and left bundle branches 812, 813 and Purkinje fibers 814, 815. In other examples, other conduction system leads can be located lower on the septum to target the right or left bundle branches 812, 813, in certain examples, respectively from the RV 807 or the LV 809 or one more locations in the coronary vasculature proximate the septal wall. Each lead can be configured to position one or more electrodes or other sensors at various locations in or near the heart 805 to detect physiologic information or provide one or more therapies or stimulation.

The first lead 820, positioned in the RA 806, includes a first tip electrode 821 located at or near the distal end of the first lead 820 and a first ring electrode 822 located near the first tip electrode 821. The second lead 825 (dashed), positioned in the RV 807, includes a second tip electrode 826 located at or near the distal end of the second lead 825 and a second ring electrode 827 located near the second tip electrode 826. The third lead 830, positioned in the coronary vein 816 over the LV 809, includes a third tip electrode 831 located at or near the distal end of the third lead 830 and a third ring electrode 832 located near the third tip electrode 831. The fourth lead 835, positioned in the RV 807 near the His bundle 811, includes a fourth tip electrode 836 located at or near the distal end of the fourth lead 835 and a fourth ring electrode 837 located near the fourth tip electrode 836. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 825 includes a first defibrillation coil electrode 828 located near the distal end of the second lead 825 in the RV 807 and a second defibrillation coil electrode 829 located a distance from the distal end of the second lead 825, such as for placement in or near the superior vena cava (SVC) 817.

Different CRM devices include different number of leads and lead placements. For examples, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing often use lead ports designated for LV or RV leads to deliver stimulation to the His bundle 811.

Sensing electrical activity from the RV 807 near the His bundle 811 is different than sensing electrical activity from the apex of the RV 807 or LV and invites separate challenges. In devices having the first lead 820 in the RA 806 and the fourth lead 835 in the RV 807 near the His bundle 811, atrial activity can be mistakenly sensed or determined by the fourth lead 835 as ventricular activity, potentially double counting ventricular activation. To address such issues, ventricular sensing thresholds can be raised or ventricular blanking periods can be extended to avoid oversensing atrial activity at the His bundle 811 as ventricular activity, such as disclosed in the commonly assigned Burr U.S. application Ser. No. 17/541,075, titled “Ventricular Blanking Periods After Atrially Sensed Beats,” herein incorporated by reference in its entirety, including its disclosure of ventricular blank periods following atrial sense events.

In an example, the implantable medical device 801 can include an antenna, such as in the header 802, configured to enable communication with an external system and one or more electronic circuits (e.g., an assessment circuit, etc.) in a hermetically sealed housing (CAN).

The implantable medical device 801 may include an insertable cardiac monitor, pacemaker, defibrillator, cardiac resynchronization therapy device, or other subcutaneous implantable medical device or cardiac rhythm management (CRM) device configured to be implanted in a chest of a subject, having one or more leads to position one or more electrodes or other sensors at various locations in or near the heart 805, such as in one or more of the atria or ventricles. Separate from, or in addition to, the one or more electrodes or other sensors of the leads, the implantable medical device system 800 can include one or more electrodes or other sensors (e.g., a pressure sensor, an accelerometer, a gyroscope, a microphone, etc.) powered by a power source in the implantable medical device 801. The one or more electrodes or other sensors of the leads, the implantable medical device 801, or a combination thereof, can be configured to detect physiologic information from, or provide one or more therapies or stimulation to, the patient.

Implantable devices can additionally include a leadless cardiac pacemaker, small (e.g., smaller than traditional implantable devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart 805 without traditional lead or implantable device complications (e.g., required incision and pocket, complications associated with lead placement, breakage, or migration, etc.). In certain examples, a leadless cardiac pacemaker can have more limited power and processing capabilities than a traditional CRM device; however, multiple leadless cardiac pacemaker devices can be implanted in or about the heart to detect physiologic information from, or provide one or more therapies or stimulation to, one or more chambers of the heart. The multiple leadless cardiac pacemaker devices can communicate between themselves, or one or more other implanted or external devices.

The implantable medical device 801 can include one or more electronic circuits configured to sense one or more physiologic signals, such as an electrogram or a signal representing mechanical function of the heart 805. In certain examples, the housing may function as an electrode such as for sensing or pulse delivery. For example, an electrode from one or more of the leads may be used together with the housing such as for unipolar sensing of an electrogram or for delivering one or more pacing pulses. A defibrillation electrode (e.g., the first defibrillation coil electrode 828, the second defibrillation coil electrode 829, etc.) may be used together with the housing to deliver one or more cardioversion/defibrillation pulses.

In an example, the implantable medical device 801 can sense impedance such as between electrodes located on one or more of the leads or the housing. The implantable medical device 801 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the implantable medical device 801 can be configured to inject current between an electrode on one or more of the first, second, third, or fourth leads 820, 825, 830, 835 and the housing, and to sense the resultant voltage between the same or different electrodes and the housing.

The implantable medical device 801 can integrate one or more other physiologic sensors to sense one or more other physiologic signals, such as one or more of heart rate, heart rate variability, thoracic or intrathoracic impedance, intracardiac impedance, arterial pressure, pulmonary artery pressure, RV pressure, LV coronary pressure, coronary blood temperature, blood oxygen saturation, one or more heart sounds, physical activity or exertion level, physiologic response to activity, posture, respiration, body weight, or body temperature. The arrangement and functions of these leads and electrodes are described above by way of example and not by way of limitation. Depending on the need of the patient and the capability of the implantable device, other arrangements and uses of these leads and electrodes are contemplated.

FIG. 9 illustrates example implantable medical devices, including a subcutaneous implantable cardioverter defibrillator 901, a leadless cardiac pacemaker 902 including tines for placement, and an insertable cardiac monitor 903.

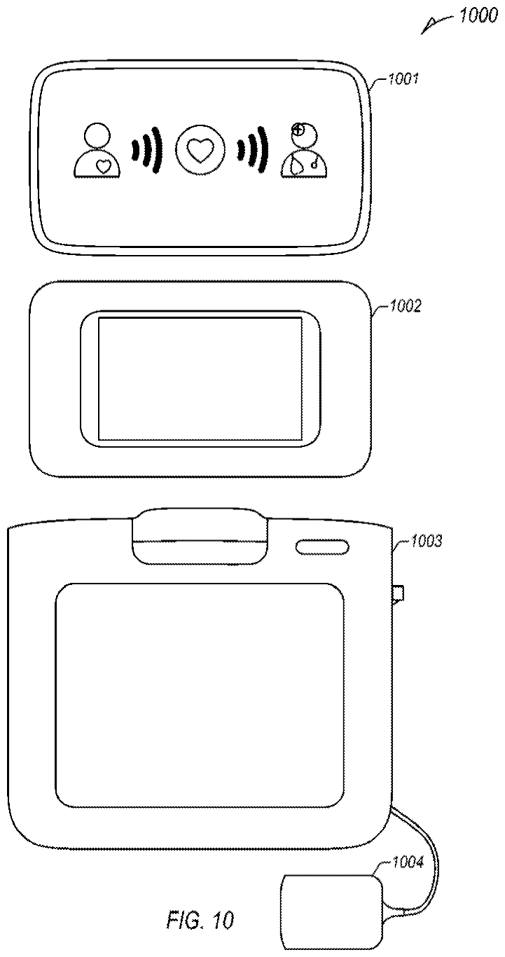

FIG. 10 illustrates example remote patient management systems 1000 for communication with an ambulatory medical device, such as for receiving information from or providing information to, including programming instructions, one or more ambulatory medical devices. The example remote patient management systems 1000 include a first remote patient management device 1001 (e.g., a LATITUDE™ NXT Remote Patient Management System) for at-home monitoring and RF telemetry capabilities through one or more communication circuits with an ambulatory medical device and communication to a cloud-based server or clinician programming environment through a network connection, a second remote patient management device 1002 (e.g., an EMBLEM™ S-ICD Programmer) with RF telemetry capabilities through one or more communication circuits and an optional external telemetry wand for communication with an ambulatory medical device, and a third remote patient management system 1003 (e.g., a LATITUDE™ Programming System, Model 3300, etc.) with RF telemetry capabilities through one or more communication circuits and an external telemetry wand 1004 (e.g., a Model 6395 Telemetry Wand, etc.) including an external telemetry coil configured for inductive communication with a corresponding telemetry coil of an implantable medical device. Although not illustrated herein, the remote patient management systems 1000 can include one or more other remote patient management systems, such as one or more other LATITUDE™ Programming systems, a remote patient monitoring application for a mobile device of a patient or other caregiver, etc. Each type of remote patient monitoring or management system has different capabilities and in certain examples permissions with respect to different programming instructions or features.

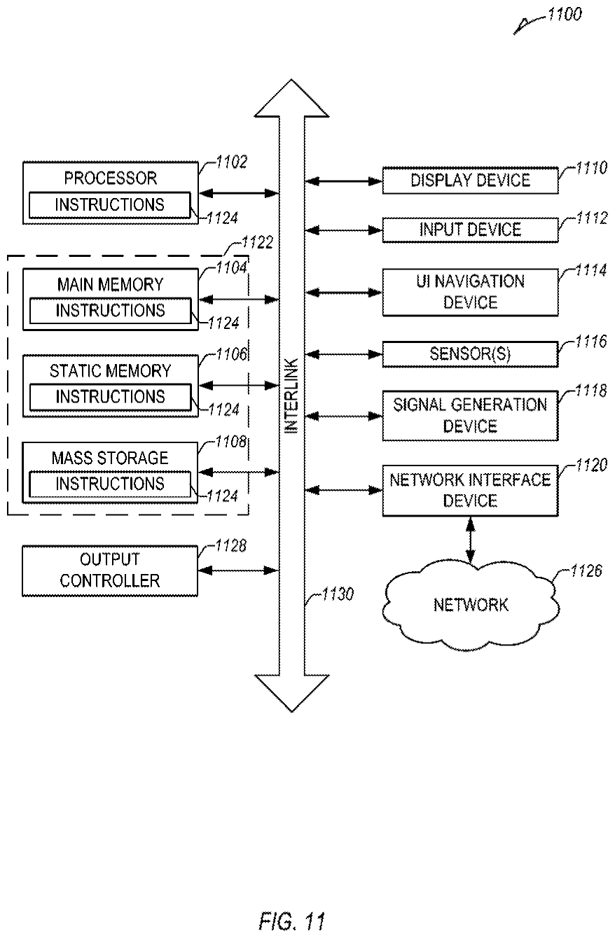

FIG. 11 illustrates a block diagram of an example machine 1100 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 1100. Circuitry (e.g., processing circuitry, an assessment circuit, etc.) is a collection of circuits implemented in tangible entities of the machine 1100 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to perform a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to perform portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1100 follow.

In alternative embodiments, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may function as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 1100 (e.g., computer system) may include a hardware processor 1102 (e.g., a central processing unit (CPU), a graphics processing unit (GPU), a hardware processor core, or any combination thereof), a main memory 1104, a static memory 1106 (e.g., memory or storage for firmware, microcode, a basic-input-output (BIOS), unified extensible firmware interface (UEFI), etc.), and mass storage 1108 (e.g., hard drive, tape drive, flash storage, or other block devices) some or all of which may communicate with each other via an interlink 1130 (e.g., bus). The machine 1100 may further include a display unit 1110, an alphanumeric input device 1112 (e.g., a keyboard), and a user interface (UI) navigation device 1114 (e.g., a mouse). In an example, the display unit 1110, input device 1112, and UI navigation device 1114 may be a touch screen display. The machine 1100 may additionally include a signal generation device 1118 (e.g., a speaker), a network interface device 1120, and one or more sensors 1116, such as a global positioning system (GPS) sensor, compass, accelerometer, or one or more other sensors. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1102, the main memory 1104, the static memory 1106, or the mass storage 1108 may be, or include, a machine-readable medium 1122 on which is stored one or more sets of data structures or instructions 1124 (e.g., software) embodying or utilized by any one or more of the techniques or functions described herein. The instructions 1124 may also reside, completely or at least partially, within any of registers of the processor 1102, the main memory 1104, the static memory 1106, or the mass storage 1108 during execution thereof by the machine 1100. In an example, one or any combination of the hardware processor 1102, the main memory 1104, the static memory 1106, or the mass storage 1108 may constitute the machine-readable medium 1122. While the machine-readable medium 1122 is illustrated as a single medium, the term “machine-readable medium” may include a single medium or multiple media (e.g., a centralized or distributed database, and/or associated caches and servers) configured to store the one or more instructions 1124.

The term “machine-readable medium” may include any medium that is capable of storing, encoding, or carrying instructions for execution by the machine 1100 and that cause the machine 1100 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1124 may be further transmitted or received over a communications network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1120 may include one or more physical jacks (e.g., Ethernet, coaxial, or phone jacks) or one or more antennas to connect to the communications network 1126. In an example, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 1100, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system to enable remote optimization of pacing parameters for conduction system pacing to optimize resources of an ambulatory medical device, comprising:
a signal receiver circuit configured to receive physiologic information from a patient; and
an assessment circuit configured to:
capture a reference paced template for the patient based on the received physiologic information and information from a clinician during an in-clinic programming session with respect to a first set of conduction system pacing parameters;
detect an ambulatory paced beat using the received physiologic information subsequent to the in-clinic programming session;
compare physiologic information from the ambulatory paced beat to the reference paced template to determine a difference between the physiologic information from the ambulatory paced beat and the reference paced template; and
provide a control signal to control or adjust a process or a function of the ambulatory medical device based on the difference between the physiologic information from the ambulatory paced beat and the reference paced template and a match threshold to optimize resources of the ambulatory medical device.

2. The system of claim 1, comprising the ambulatory medical device, wherein the ambulatory medical device comprises the signal receiver circuit and the assessment circuit,
wherein the ambulatory medical device includes an implantable medical device configured to deliver conduction system pacing to the patient,
wherein to provide the control signal to control or adjust the process or the function of the ambulatory medical device includes to control or adjust a conduction system pacing parameter including at least one of a voltage, atrioventricular delay, polarity, or pulse width of a conduction system pacing stimulus.

3. The system of claim 2, wherein the signal receiver circuit of the implantable medical device is configured to receive the physiologic information from an electrode coupled to the implantable medical device, wherein the electrode is configured to sense cardiac electrical information from a heart of the patient and to deliver the conduction system pacing stimulus to a conduction pathway of the heart of the patient.

4. The system of claim 3, wherein the physiologic information includes at least one of nearfield or farfield cardiac electrical information or cardiac acceleration information of the heart of the patient.

5. The system of claim 1, wherein the assessment circuit comprises or is coupled to:
a therapy circuit configured to generate a pacing stimulus; and
a memory circuit configured to store information, including the reference paced template,
wherein the reference paced template comprises at least one of farfield cardiac electrical information, nearfield cardiac electrical information, cardiac acceleration information, a relationship between different cardiac electrical information, a relationship between different cardiac acceleration information, a relationship between cardiac electrical and cardiac acceleration information, or a relationship between pacing information and one or more of cardiac electrical information and cardiac acceleration information,
wherein cardiac acceleration information comprises a heart sound including at least one of an S1, S2, S3, or S4 heart sound.

6. The system of claim 5, wherein the reference paced template includes a morphology of the cardiac electrical information.

7. The system of claim 5, wherein the cardiac electrical information includes at least one of atrial information or ventricular information.

8. The system of claim 1, wherein to determine the difference between the physiologic information from the ambulatory paced beat and the reference paced template, the assessment circuit is configured to:
determine a correlation between the physiologic information from the ambulatory paced beat and the reference paced template; or
determine a difference between a feature of the physiologic information from the ambulatory paced beat and a corresponding feature of the reference paced template.

9. The system of claim 1, wherein to control or adjust the process or the function of the ambulatory medical device, the assessment circuit is configured to:
adjust a conduction system pacing parameter of the ambulatory medical device based on the difference between the physiologic information from the ambulatory paced beat and the reference paced template exceeding the match threshold; and
maintain the conduction system pacing parameter of the ambulatory medical device based on the difference between the physiologic information from the ambulatory paced beat and the reference paced template being within the match threshold.

10. The system of claim 9, wherein to adjust the conduction system pacing parameter, the assessment circuit is configured to adjust, sequentially:
a voltage of a conduction system pacing stimulus over a number of respective beats to determine an adjusted voltage resulting in a match between physiologic information of the respective beat and the reference paced template based on a difference between the physiologic information of the respective beat and the reference paced template being within the match threshold; and
for each voltage resulting in the match, an atrioventricular delay of the conduction system pacing stimulus over a number of respective beats to determine an adjusted atrioventricular delay resulting in a match between physiologic information of the respective beat and the reference paced template based on a difference between the physiologic information of the respective beat and the reference paced template within the match threshold,
wherein the assessment circuit is configured to program the voltage and atrioventricular delay having a smallest difference between the physiologic information of the respective beat and the reference paced template.

11. The system of claim 1, wherein to adjust the conduction system pacing parameter, the assessment circuit is configured to adjust, sequentially:
a voltage, a polarity, and a pulse width of a conduction system pacing stimulus over a number of respective beats to determine pacing parameters resulting in a match between physiologic information of the respective beat and the reference paced template based on a difference between the physiologic information of the respective beat and the reference paced template within the match threshold,
wherein the assessment circuit is configured, for each set of pacing parameters resulting in the match, to adjust:
an atrioventricular delay of the conduction system pacing stimulus over a number of respective beats to determine an adjusted atrioventricular delay resulting in a match between physiologic information of the respective beat and the reference paced template based on a difference between the physiologic information of the respective beat and the reference paced template within the match threshold,
wherein the assessment circuit is configured to program the pacing parameters having a closest match between the physiologic information of the respective beat and the reference paced template.

12. A method to enable remote optimization of pacing parameters for conduction system pacing to optimize resources of an ambulatory medical device, comprising:
receiving physiologic information from a patient using a signal receiver circuit;
capturing, using an assessment circuit, a reference paced template for the patient based on the received physiologic information and information from a clinician during an in-clinic programming session with respect to a first set of conduction system pacing parameters;
detecting, using the assessment circuit, an ambulatory paced beat using the received physiologic information subsequent to the in-clinic programming session;
comparing, using the assessment circuit, physiologic information from the ambulatory paced beat to the reference paced template to determine a difference between the physiologic information from the ambulatory paced beat and the reference paced template; and
providing, using the assessment circuit, a control signal to control or adjust a process or a function of the ambulatory medical device based on the difference between the physiologic information from the ambulatory paced beat and the reference paced template and a match threshold to optimize resources of the ambulatory medical device.

13. The method of claim 12, wherein providing the control signal to control or adjust the process or the function of the ambulatory medical device includes controlling or adjusting a conduction system pacing parameter including at least one of a voltage, atrioventricular delay, polarity, or pulse width of a conduction system pacing stimulus,
wherein the signal receiver circuit and the assessment circuit are components of an implantable medical device configured to deliver conduction system pacing to the patient,
wherein receiving the physiologic information from the patient comprises receiving the physiologic information from an electrode coupled to the implantable medical device, wherein the electrode is configured to sense cardiac electrical information from a heart of the patient and to deliver the conduction system pacing stimulus to a conduction pathway of the heart of the patient.

14. The method of claim 13, wherein the physiologic information includes at least one of nearfield or farfield cardiac electrical information or cardiac acceleration information of the heart of the patient.

15. The method of claim 12, wherein the reference paced template comprises at least one of farfield cardiac electrical information, nearfield cardiac electrical information, cardiac acceleration information, a relationship between different cardiac electrical information, a relationship between different cardiac acceleration information, a relationship between cardiac electrical and cardiac acceleration information, or a relationship between pacing information and one or more of cardiac electrical information and cardiac acceleration information,
wherein cardiac acceleration information comprises a heart sound including at least one of an S1, S2, S3, or S4 heart sound.

16. The method of claim 15, wherein the reference paced template includes a morphology of the cardiac electrical information.

17. The method of claim 12, wherein determining the difference between the physiologic information from the ambulatory paced beat and the reference paced template includes:
determining a correlation between the physiologic information from the ambulatory paced beat and the reference paced template; or
determining a difference between a feature of the physiologic information from the ambulatory paced beat and a corresponding feature of the reference paced template.

18. The method of claim 12, wherein controlling or adjusting the process or the function of the ambulatory medical device comprises:
adjusting a conduction system pacing parameter of the ambulatory medical device based on the difference between the physiologic information from the ambulatory paced beat and the reference paced template exceeding the match threshold; and
maintaining the conduction system pacing parameter of the ambulatory medical device based on the difference between the physiologic information from the ambulatory paced beat and the reference paced template being within the match threshold.

19. The method of claim 18, wherein adjusting the conduction system pacing parameter comprises adjusting, sequentially:
a voltage of a conduction system pacing stimulus over a number of respective beats to determine an adjusted voltage resulting in a match between physiologic information of the respective beat and the reference paced template based on a difference between the physiologic information of the respective beat and the reference paced template being within the match threshold; and
for each voltage resulting in the match, an atrioventricular delay of the conduction system pacing stimulus over a number of respective beats to determine an adjusted atrioventricular delay resulting in a match between physiologic information of the respective beat and the reference paced template based on a difference between the physiologic information of the respective beat and the reference paced template within the match threshold,
wherein the method includes programming the voltage and atrioventricular delay having a smallest difference between the physiologic information of the respective beat and the reference paced template.

20. The method of claim 12, wherein adjusting the conduction system pacing parameter to optimize resources of an ambulatory medical device comprises adjusting, sequentially:
a voltage, a polarity, and a pulse width of a conduction system pacing stimulus over a number of respective beats to determine pacing parameters resulting in a match between physiologic information of the respective beat and the reference paced template based on a difference between the physiologic information of the respective beat and the reference paced template within the match threshold,
wherein the method includes, for each set of pacing parameters resulting in the match, adjusting:
an atrioventricular delay of the conduction system pacing stimulus over a number of respective beats to determine an adjusted atrioventricular delay resulting in a match between physiologic information of the respective beat and the reference paced template based on a difference between the physiologic information of the respective beat and the reference paced template within the match threshold,
wherein the method includes programming the pacing parameters having a closest match between the physiologic information of the respective beat and the reference paced template.
